(19) United States
(12) Patent Application Publication
(10) Pub. No.: US 2025/0170237 A1
(43) Pub. Date: May 29, 2025

(54) MURAMYL DIPEPTIDES AND PROCESS FOR PREPARATION THEREOF

(71) Applicant: Council of Scientific and Industrial Research, New Delhi (IN)

(72) Inventors: Halmuthur Mahabalarao Sampath KUMAR, Hyderabad (IN); Bonam Srinivasa REDDY, Hyderabad (IN); Paturu Rama Subba REDDY, Hyderabad (IN); Atla SANDEEP, Hyderabad (IN)

(21) Appl. No.: 18/841,573

(22) PCT Filed: Feb. 24, 2023
(86) PCT No.: PCT/IN2023/050177

§ 371 Date: Aug. 26, 2024

(30) Foreign Application Priority Data
Feb. 25, 2022 (IN) 202211011022

(51) Int. Cl. A61K 39/39; A61K 39/00; C07K 1/06; C07K 1/10; C07K 5/062

(52) U.S. Cl. CPC A61K 39/39; C07K 1/065; C07K 1/10; C07K 5/06026; A61K 2039/5555

(57) ABSTRACT

The present invention relates to Muramyl dipeptide compounds having adjuvant activity. The present invention also discloses the process for the preparation of Muramyl dipeptide compound and their intermediates. The immuno-modulating properties of the Muramyl dipeptide compound and their use as NOD2 agonistic adjuvants in vaccine formulations is also disclosed.

### FIELD OF THE INVENTION

The present invention relates to Muramyl dipeptide compounds having adjuvant activity. The present invention particularly relates to Muramyl dipeptide compound of general Formula-I.

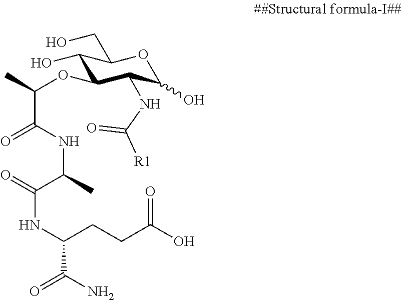

The present invention also relates to the process for the preparation of Muramyl dipeptide compound of general Formula-I having adjuvant activity and their intermediates. The present invention also discloses the immuno-modulating properties of the Muramyl dipeptide compound and their use as NOD2 agonistic adjuvants in vaccine formulations.

### BACKGROUND OF THE INVENTION

Several naturally occurring glycoprotein's and glycopeptides exhibit powerful immune modulatory properties and often used as adjuvants for vaccines, synthetic vaccines and in cancer immunotherapy. Of particular interest are the glycoproteins/peptides bearing glucosamine moiety which play important role in regulating the immune system. While some of these glycoproteins/peptides are tumor cell or tumor surface markers. Many N-acyl glycopreteins are the part of peptidoglycan moiety of several gram positive and gram negative bacteria and hence are highly immunogenic in nature. Muramyl dipeptide (MDP, N-acetylmuramyl-L-alanyl-D-isoglutamine)-is a synthetic immune-reactive peptide consisting of N-acetyl muramic acid attached to a short amino acid chain of L-Ala-D-isoGln. It was first identified in bacterial cell wall peptidoglycan as an active component in Freund's Complete Adjuvant (FCA). In 1974, MDP was discovered to be the minimal structure required for the efficacy of FCA, -one of the most potent and widely used adjuvants in animal experimental models. Muramyl dipeptide derivatives have been proved to show significant immunomodulatory properties, via its cognate PRR (Pattern Recognition Receptor), nucleotide-binding oligomerization domain 2 (NOD2) (Girardin S. et al., 2003. J Biol Chem. 278(11): 8869-72; F. Coulombe et al, 2012 PloS ONE, 7 (5): Article ID e36734). Muramyl dipeptides activate macrophages and other cells of the immune system to kill cancer cells (7. Jakopin, 2013. Current Medicinal Chemistry, 20 (16): 2068-2079; Ogawa et. al., 2011. CurrBioact Compd. 7(3): 180-197), however, it is also reported to be pyrogenic in nature. In order to reduce its pyrogenic effect, and improve its efficacy, several MDP derivatives have been designed, synthesized and tested till date. There are many reported inventions or publications related to the synthesis of Muramyl dipeptide and its derivatives (Namba et al., 1997. Vaccine, 15(4):405-13; WO1996001645; U.S. Pat. No. 4,395,399, 7,173,107B2).

N-glycolyl MDP (N-glycolyl glucosaminyl-N-acetylmuramyl-L-alanyl-D-isoglutamine), Murabutide are few examples of Muramyl dipeptide derivative, which, exhibit higher immunoadjuvant activity and less pyrogenic effect, compared to MDP. Several other derivatives of MDP are reported to be better immuno-therapeutics compared to parent compound (Andronova T. M., et al., 1991. Review. Immunology 4,1). Hence, this molecule is been widely used in immunotherapeutic approaches, especially to treat chronic infections, autoimmune diseases and cancer (L. I. Rostovtseva et al., 1981. Russian Journal of Bioorganic Chemistry, 7 (12):1843-1858). MDP-based compound GMDP also known as Likopid™ is the first immunotherapeutic of the muramyl glycopeptide structural class, introduced to the clinical practice. Likopid™ was developed and registered by a Russian company Peptek as an immunotherapeutic with broad applicability, e.g. immune stimulation and prevention of infections complicating post-traumatic, post-operative, post-chemotherapeutic and post-radiotherapeutic patient hood. Other areas are the treatment of infectious diseases, like tuberculosis, human cervical papillomavirus, ophthalmic herpetic infections, psoriasis and treatment of ulcerous and inflammation processes (WO2007045192). Below shown Formula-X represents the general chemical structure of muramyl dipeptides (MDPs). The MDP and its derivatives are known to exhibit potential applications in biological studies as, for example, NMR probes, imaging or affinity labels. The MDP derivatives may be used as the building blocks for making peptidoglycans in vitro or in vivo, which may be used to modify the cell wall of bacterial cells or modulate an innate immune response. (WO2016172615 and US2016/029026)

Below shown Formula-X represents the general chemical structure of muramyl dipeptides (MDPs) and Glycolyl MDP.

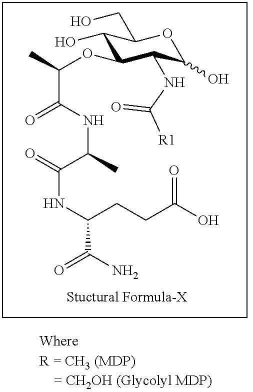

WO1996001645A1 relates to the use of Muramyl peptide compounds, particularly N-acetyl-D-glucosaminyl-(pi-4)-N-acetylmuramyl-L-alanyl-D-isoglutamine (MDP), for the treatment of inflammatory dermatological conditions such as psoriasis and in the treatment of immune-related diseases of the skin and mucous membranes.

U.S. Pat. No. 7,173,107B2 discloses glycopeptides and preparation thereof, which covers the stereospecific synthesis of a glycopeptide following a triplyorthogonal protection scheme in particular, the synthesis of N-acetylglucosaminyl-P-[1,4]-N-acetylmuramylmonopeptide and derivatives thereof. The glycopeptides are shown to be useful for the preparation of MDP and related compounds having a glucosaminyl-P- [1,4]-N-acetylmuramic acid disaccharide core.

WO2007045192 relates to glucosaminylmuramic acid (2-amino-2-deoxy-P-D-gluco-pyranosyl-(1 4)-N-acetylmuramic acid) derivatives, method of their synthesis, and their use for the synthesis of glucosaminylmuramyl glycopeptides, i.e. disaccharide analogues of muramyl glycopeptides. U.S. Pat. No. 4,395,399 discloses different glycopeptides and their preparations.

However, despite improvements seen in several newly developed MDP derivatives, yet there is a continuous need for novel compounds with further reduced side-effects and increased adjuvant activity for the purpose of either therapeutic or prophylactic use in vaccine formulations. As adjuvant action is antigen specific, in order to evoke qualitatively specific immune response, there is a need to generate a variety of alternate structures mimicking peptidoglycan that are more efficacious and less toxic with high degree of complementarity with specific antigens. Hence it is critical to focus on the balance between efficacy and toxicity while designing or synthesizing new compounds. Development of more efficacious and improved peptidoglycan adjuvants, with reduced side effects associated with known MDP derivatives, is highly needed. In view of the importance of Glucosamine derived peptidoglycan as immune modulators, the present invention discloses a novel muramyl dipeptide compound and a concise process for the synthesis of the Muramyl dipeptide compounds.

### OBJECTIVE OF THE INVENTION

The primary object of the invention is to provide Muramyl dipeptide compounds having adjuvant activity. Another objective of the present invention is to provide a concise process for the preparation of Muramyl dipeptide compounds.

Yet another objective of the present invention is to provide novel intermediates compounds for the synthesis Muramyl dipeptide compounds.

A further objective of the present invention is to provide safe use of Muramyl dipeptide compounds as adjuvants with selected antigens for pharmaceutical preparations and vaccine formulations.

Yet another objective of the present invention is to provide vaccine formulations comprising Muramyl dipeptide compounds with selected antigens.

### SUMMARY OF THE INVENTION

Accordingly, the present invention provides a muramyl dipeptide compound of general formula I,

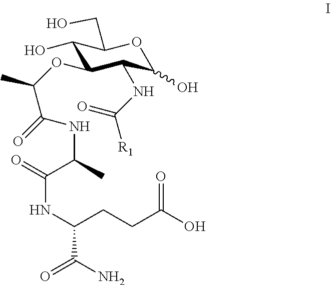

Wherein, R₁ is selected from substituted or unsubstituted (C₃-C₆) cycloalkyl, 3 to 6 membered heterocycloalkyl with one or more hetero atoms selected from Nitrogen and Oxygen or

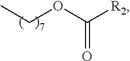

R₂ is selected from group consisting of C₁ to C₁₈ aliphatic chain.

In an embodiment of the present invention, R₁ is selected from the group consisting of

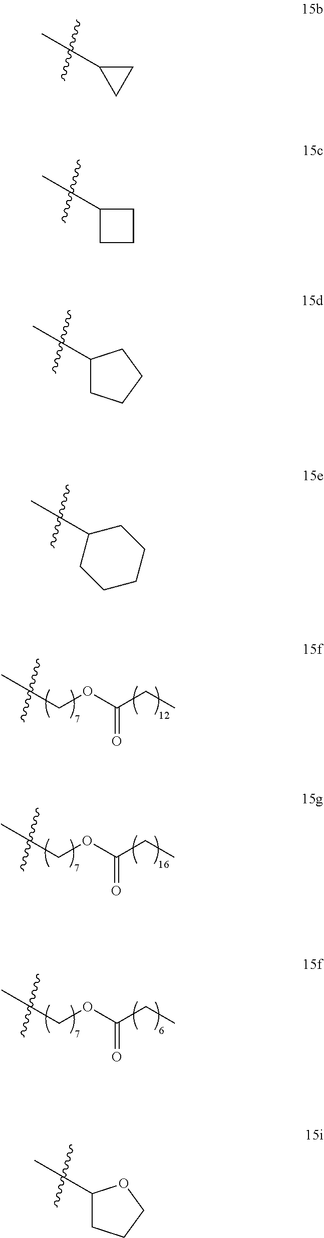

In a preferred embodiment of the present invention the compound is selected from the group consisting of;
(4R)-5-amino-4-((2S)-2-((2R)-2-(((3R,4R,5S,6R)-3-(cyclopropanecarboxamido)-2,5-dihydroxy-6-(hydroxymethyl)tetrahydro-2H-pyran-4-yl)oxy)propanamido)propanamido)-5-oxopentanoic acid (15b),
(4R)-5-amino-4-((2S)-2-((2R)-2-(((3R,4R,5S,6R)-3-(cyclobutanecarboxamido)-2,5-dihydroxy-6-(hydroxymethyl)tetrahydro-2H-pyran-4-yl)oxy)propanamido)propanamido)-5-oxopentanoic acid (15c),
(4R)-5-amino-4-((2S)-2-((2R)-2-(((3R,4R,5S,6R)-3-(cyclopentanecarboxamido)-2,5-dihydroxy-6-(hydroxymethyl)tetrahydro-2H-pyran-4-yl)oxy)propanamido)propanamido)-5-oxopentanoic acid (15d),
(4R)-5-amino-4-((2S)-2-((2R)-2-(((3R,4R,5S,6R)-3-(cyclohexanecarboxamido)-2,5-dihydroxy-6-(hydroxymethyl)tetrahydro-2H-pyran-4-yl)oxy)propanamido)propanamido)-5-oxopentanoic acid (15e),
(4R)-5-amino-4-((2S)-2-((2R)-2-(((3R,4R,5S,6R)-2,5-dihydroxy-6-(hydroxymethyl)-3-(8-(tetradecanoyloxy)octanamido)tetrahydro-2H-pyran-4-yl)oxy)propanamido)propanamido)-5-oxopentanoic acid (15f),
(4R)-5-amino-4-((2S)-2-((2R)-2-(((3R,4R,5S,6R)-3-(8-(dodecanoyloxy)octanamido)-2,5-dihydroxy-6-(hydroxymethyl)tetrahydro-2H-pyran-4-yl)oxy)propanamido)propanamido)-5-oxopentanoic acid (15 g),
(4R)-5-amino-4-((2S)-2-((2R)-2-(((3R,4R,5S,6R)-2,5-dihydroxy-6-(hydroxymethyl)-3-(8-(octanoyloxy)octanamido)tetrahydro-2H-pyran-4-yl)oxy)propanamido)propanamido)-5-oxopentanoic acid (15h) and
(4R)-5-amino-4-((2S)-2-((2R)-2-(((3R,4R,5S,6R)-2,5-dihydroxy-6-(hydroxymethyl)-3-(tetrahydrofuran-2-carboxamido)tetrahydro-2H-pyran-4-yl)oxy)propanamido)propanamido)-5-oxopentanoic acid (15i).

In an embodiment the present invention provides an intermediate compound of general formula II,

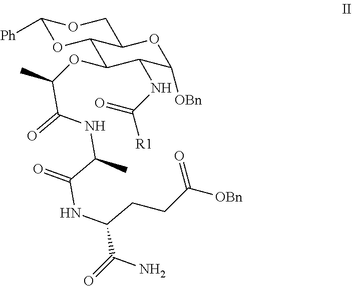

wherein R₁ is selected from substituted or unsubstituted (C₃-C₆) cycloalkyl, 3 to 6 membered heterocycloalkyl with one or more hetero atoms selected from nitrogen and oxygen or,

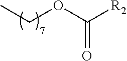

wherein R2 is selected from group consisting of C₁ to C₁₈ aliphatic chain.

The present invention also provides a process for preparing the muramyl dipeptide compound of general formula I,

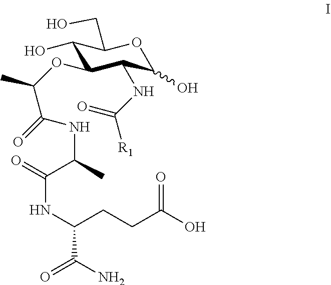

wherein R₁ is selected from substituted or unsubstituted alkyl, (C₃-C₆) cycloalkyl, 3 to 6 membered heterocycloalkyl with one or more hetero atoms selected from nitrogen and oxygen or, O

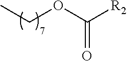

wherein R₂ is selected from group consisting of C₁ to C₁₈ aliphatic chain, comprising the steps of:
a) diazotransfer of compound 7 with trifluoromethanesulfonic azide to obtain (3R,4R,5S,6R)-3-azido-6-(hydroxymethyl)tetrahydro-2H-pyran-2,4,5-triol (compound 8);

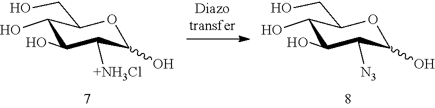

b) anomeric benzylation of compound 8 in presence of a Lewis acid to obtain (2R,3S,4R,5R,6S)-5-azido-6-(benzyloxy)-2-(hydroxymethyl)tetrahydro-2H-pyran-3,4-diol (compound 9);

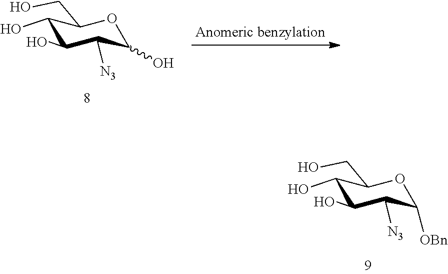

c) benzylidene protection of compound 9 in presence of an organic acid with benzldehyde dimethylacetal to obtain (2R,4aR,6S,7R,8R,8aS)-7-azido-6-(benzyloxy)-2-phenylhexahydropyrano[3,2-d][1,3]dioxin-8-ol (compound 10);

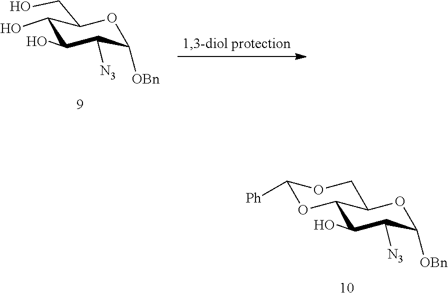

d) O-alkylation of azide compound-10 in presence of a metal hydride with S-(2)-Chloropropionic acid to obtain (R)-2-(((2R,4aR,6S,7R,8R,8aS)-7-azido-6-(benzyloxy)-2-phenylhexahydropyrano[3,2-d][1,3]dioxin-8-yl)oxy)propanoic acid (compound 11);

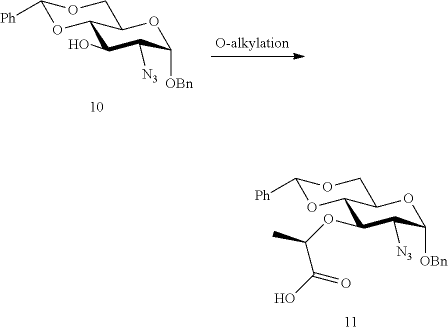

e) peptide coupling of compound 11 with L-alanyl-D-isoglutamine benzyl ester trifluoroacetate (6) to obtain (R)-benzyl 5-amino-4-((S)-2-((R)-2-(((2R,4aR,6S,7R,8R,8aS)-7-azido-6-(benzyloxy)-2-phenylhexahydropyrano[3,2-d][1,3]dioxin-8-yl)oxy)propanamido) propanamido)-5-oxopentanoate (compound 12);

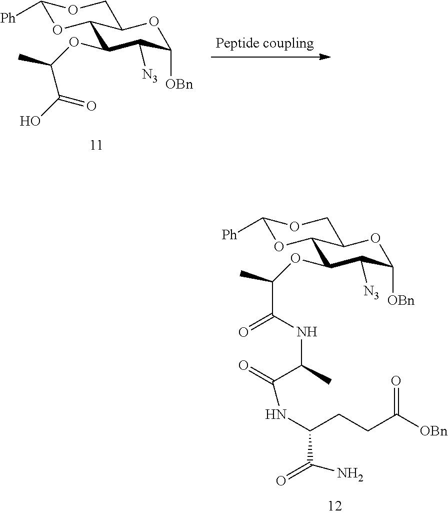

f) reduction of compound 12 with triphenylphosphine to obtain benzyl (R)-5-amino-4-((S)-2-((R)-2-(((2R,4aR,6S,7R,8R,8aS)-7-amino-6-(benzyloxy)-2-phenylhexahydropyrano[3,2-d][1,3]dioxin-8-yl)oxy)propanamido)propanamido)-5-oxopentanoate (13);

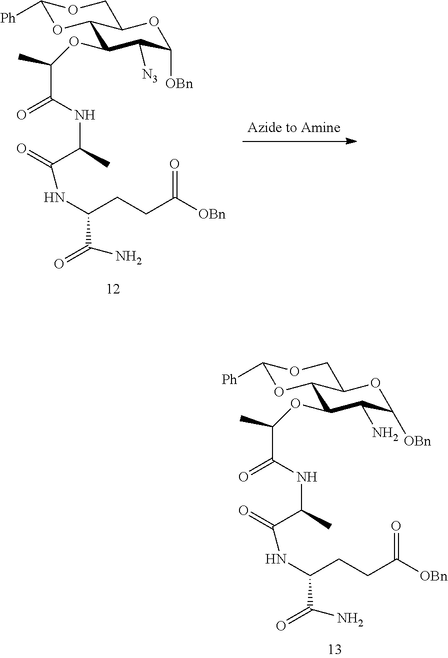

g) acid and amine coupling of compound 13 with cycloalkyl carboxylic acid or mixed ester carboxylic acid C(O)R₁ wherein R₁ is selected from substituted or unsubstituted (C₃-C₆) cycloalkyl, 3 to 6 membered heterocycloalkyl with one or more hetero atoms selected
   from nitrogen and oxygen or

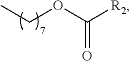

wherein R₂ is selected from group consisting of C₁ to C₁₈ aliphatic ester, in presence of a coupling agent and a base to obtain compound of formula 14;

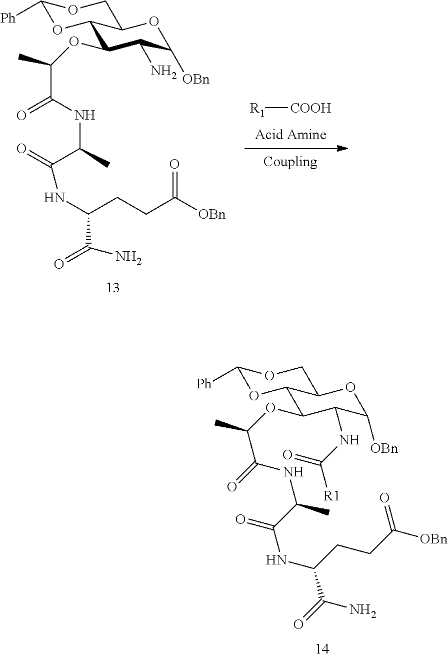

h) hydrogenolysis of compound 14 to obtain compound of formula-I (15).

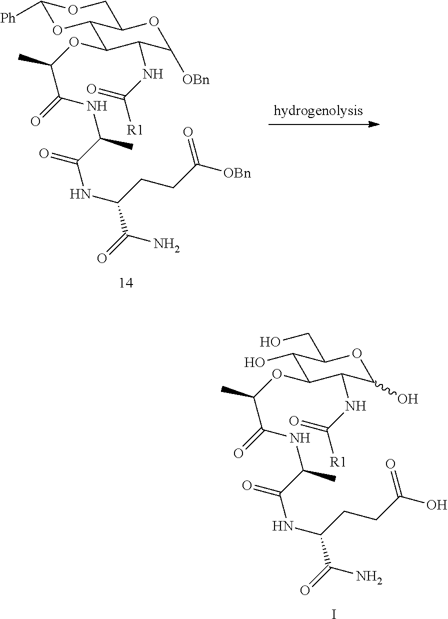

In an embodiment of the present invention, the Lewis acid used in step (b) is selected from the group consisting of borantrifluoride dietherate, AlCl₃, ZnCl₂, P-toluene sulfonic acid, Chlorosulfonic acid, Camphor sulfonic acid, Hydrochloric acid, acetyl chloride, Amberlite and zeolite, clay.

In an embodiment of the present invention, the organic acid in step (c) is selected from the group consisting of p-Toulenesulphonic acid, camphorsulphonic acid and Zinc dichloride.

In an embodiment of the present invention, the metal hydride in step (d) is selected from the group consisting of sodium hydride, potassium hydride and calcium hydride.

In an embodiment of the present invention, the coupling agent in step (e) is selected from the group consisting of EDCI/HOBt, DCC/DMAP, DIC/DMAP and T3P and the base in step (e) is diisopropylethylamine.

In an embodiment of the present invention, the coupling agent in step (g) is selected from the group consisting of EDCI/HOBt, DCC/DMAP, DIC/DMAP and T3P and the base in step (g) is diisopropylethylamine.

In an embodiment of the present invention, pharmaceutical composition comprising: a muramyl dipeptide compound of general Formula-I,

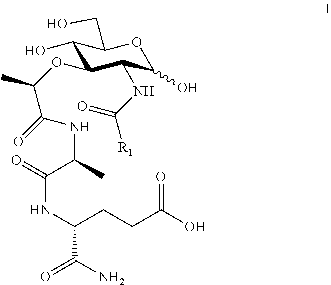

Wherein, R₁ is selected from substituted or unsubstituted (C₃-C₆) cycloalkyl, 3 to 6 membered heterocycloalkyl with one or more hetero atoms selected from Nitrogen and Oxygen or

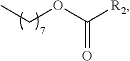

R₂ is selected from group consisting of C₁ to C₁₈ aliphatic chain and an antigen.

In a preferred embodiment of the present invention, the antigen is selected from inactivated or live attenuated infectious pathogens against mammals, their sub unit antigens either natural derived or recombinant, a conjugate vaccine antigen or a combination thereof.

Abbreviations used in the present invention process have the following meaning or have been elaborated as follows:
L-Ala-D-isoGln: L-alanyl-D-DIPEA: N,N-Diisopropylethylamine isoglutamine EDCI: 1-Ethyl-3-(3-dimethylaminopropyl)
Na₂SO₄: Sodium Sulphate carbodiimide
BF₃.E₂O: Borantrifluoro etherate THF-i-Pr₂O: Tetrahydroforan-Diisopropylether
Boc₂O: Di-tert-butyl dicarbonate DMF: Dimethylformamide
THF: Tetrahydrofuran P₂O₅: Phousphorus pentoxide
DCM: Dichloromethane HOBt: Hydroxy benzotriazole
ACN: Acetonitrile NaH: Sodium hydride
EtOAc: Ethyl acetate TFA: Trifluoroaceticacid
Et₃N: Triethylamine T3P: 1-Propanephosphonic acid anhydride
PTSA: P-tolune sulphonic acid

### BRIEF DESCRIPTION OF THE DRAWINGS

The following drawings form part of the present specification and are included to further illustrate aspects of the present disclosure. The disclosure may be better understood by reference to the drawings in combination with the detailed description of the specific embodiments presented herein.

FIG. 1 represents synthesis of dipeptide.

FIG. 2 represents synthesis of Muramyl dipeptide of general formula-I.

### DETAILED DESCRIPTION OF THE INVENTION

The present invention provides Muramyl dipeptide of general Formula-I, with reduced pyrogenicity, while maintaining a considerable effective function as vaccine adjuvants.

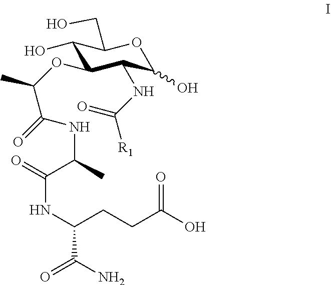

wherein, R₁ is selected from substituted or unsubstituted (C₃-C₆) cycloalkyl, 3 to 6 membered heterocycloalkyl with one or more hetero atoms selected from Nitrogen and Oxygen or

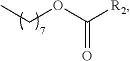

R₂ is selected from group consisting of C₁ to Cis aliphatic chain.

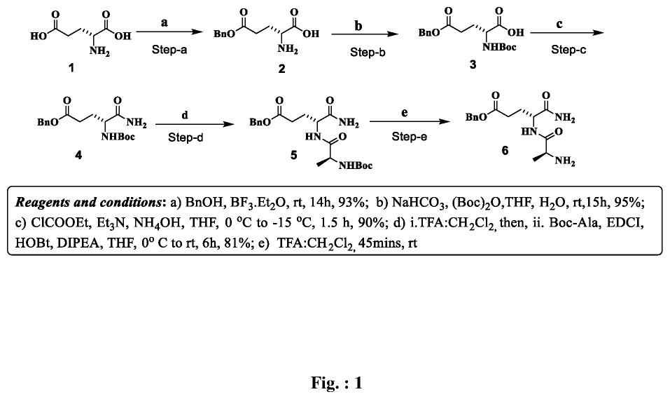

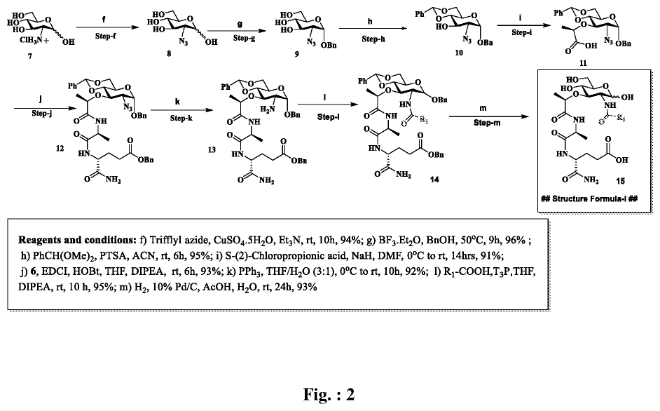

In another aspect, the present invention provides a process for preparation of Muramyl dipeptide of general Formula-I. The present process has advantages over the prior art in terms of reduced number of steps to achieve the synthesis of key intermediate obviating redundant acylation, selective of anomeric hydroxyl group, deacylation followed by activation and its subsequent deacylation, thereby generating less waste and improving the overall efficiency and yield of the process compared to previous processes, offering better atom economy. The invention relates to process for the preparation Muramyl dipeptide of general Formula-I which are depicted in FIG. 1 and FIG. 2.

L-alanyl-D-isoglutamine benzyl ester (dipeptide) obtained in FIG. 1 is used for the synthesis of Muramyl dipeptide of general Formula-I as shown in FIG. 2.

The Muramyl dipeptide of general formula-I, wherein, R₁ is selected from substituted or unsubstituted alkyl, (C₃-C₆) cycloalkyl, 3 to 6 membered heterocycloalkyl with one or more hetero atoms selected from Nitrogen and Oxygen or

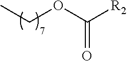

wherein R₂ is selected from group consisting of C₁ to Cis Aliphatic chain, is synthesized in FIG. 2.

In another aspect, the invention provides a process for the synthesis of muramyl dipeptide of Formula-I involving the reaction steps as depicted in above FIG-1 for the synthesis of the L-alanyl-D-isoglutamine benzyl ester and FIG. 2 for the synthesis of novel N-glycolyl peptidoglycan derivatives of Structural Formula-I.

In yet another embodiment, the invention provides a novel N-glycolyl peptidoglycan intermediate of Structural formula-II

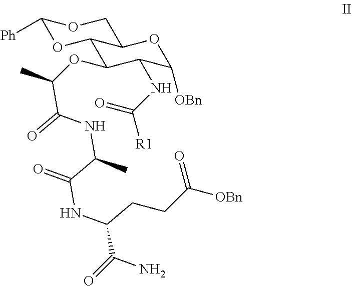

wherein R₁ is selected from substituted or unsubstituted (C₃-C₆) cycloalkyl, 3 to 6 membered heterocycloalkyl with one or more hetero atoms selected from nitrogen and oxygen or,

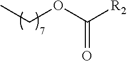

wherein R₂ is selected from group consisting of C₁ to C₁₈ aliphatic chain.

FIG. 1: Provides the process for the synthesis of dipeptide according to the following steps.

Step-a: [Compound 2] To a stirred mixture of D-Glutamic acid [Compound 1] and anhydrous sodium sulphate (Na₂SO₄) suspended in benzyl alcohol, added boron trifluoride. diethyl ether (BF₃. E₂O) and the suspension is stirred at room temperature (RT) followed by diluting with absolute THF and filtering with the aid of charcoal. Treatment with triethylamine, followed by concentration and precipitation and washing provides (R)-2-amino-5-(benzyloxy)-5-oxopentanoic acid [Compound 2].

Step-b: [Compound 3] is prepared by providing a solution of the [compound 2], adding with Di-teritiary butyl dicarbonate (Boc)₂O in dioxane and water at a low temperature (0-5° C.) and basified with sodium bicarbonate (NaHCO₃), stirring overnight. Then the solvent is removed under reduced pressure and the residue is diluted with water, and washed with Ethyl Acetate (EtOAc), adjusted to pH 2-3 with aqueous HCl solution and extracted with EtOAc followed by further processing to get(R)-5-(benzyloxy)-2-(tert-butoxycarbonyl)-5-oxopentanoic acid[Compound 3].

Step-c: [Compound 4] is synthesized by providing [Compound 3] in dry Tetrahydrofuran (THF), adding ethyl chloroformate and triethylamine at a low temperature such as 0° C. The reaction mixture is stirred at low temperature, and then cooled to low temperature (such as −15° C.) followed by addition of a methanolic solution of ammonia and further cooling to one hour at minus temperature and is added with ethyl acetate. After washing the organic phase with water followed by washing with brine solution, drying, removing the solvent in vacuum and purification)-benzyl-5-amino-4-((tert-butoxy carbonyl) amino)-5-oxopentanoate [Compound 4] is obtained.

Step-d: [Compound 5]i-Butoxy carbonyl-D-isoglutamine benzyl ester [Compound 4] is dissolved in cold trifluoroacetic acid and the resultant solution is stirred at RT and then Trifluoroacetic acid is removed and the residue is triturated with Diethyl Ether (Et₂O) to obtain oily D-isoglutamine benzyl ester trifluoroacetate. Separately, to t-butoxycarbonyl-L-alanine in dry THF, 1-Ethyl-3-(3-dimethyl aminopropyl) carbodiimide (EDCI) and Hydroxy benzotriazole (HOBt) are added and stirred at RT. To this solution, D-isoglutamine benzyl ester trifluoroacetate (dissolved in THF) is added followed by N, N-Diisopropylethylamine (DIPEA). After stirring and concentrating the residue is extracted with EtOAc, washed and dried to obtain a residue. After further washing and recrystallization it provides benzyl-5-amino-4-((5)-2-((tert-butoxycarbonyl)amino) propanamido)-5-oxopentanoate [Compound 5].

Step-e: [Compound 6]benzyl-5-amino-4-((5)-2-((tert-butoxycarbonyl)amino) propanamido)-5-oxopentanoate [Compound 5] is dissolved in cold trifluoroacetic acid and dichloromethane and the resultant solution is stirred at RT and then Trifluoroacetic acid is removed with toluene by making azeotropic mixture and the residue was triturated with Diethyl Ether (Et₂O) to obtain oily benzyl(R)-5-amino-4-((S)-2-aminopropanamido)-5-oxopentanoatetrifluoroacetate[Compound 6]

FIG. 2 provides the process for the synthesis of muramyl dipeptide of general formula-I, according to the following steps.

Step-(f): Diazo transfer of glucosamine hydrochloride[Compound 8].

Step (f) involves Diazo transfer of Formula 7 with a suitable azide transferring agent in the presence of an organic base with a suitable solvent, to obtain a compound of Formula-8 as schematically presented below;

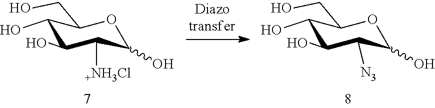

To the mechanically stirred solution of sodium azide in dry acetonitrile add triflic anhydride at 0° C. in 5Ltr RBF and kept it stirring for 30 minutes insitu triflic azide generated. To the mechanically stirred solution of glucosamine hydrochloride [Compound 7] copper sulphate. pentahydrate and amine base in the second 5Ltr RBF at 0° C. while stirring. After 15 min to the 2^{nd} RBF at 0° C. triflic azide was added then kept it rt for 10 hrs. after completion of reaction. The reaction mixture was extracted in EtOAc (3×100 ml) then combined organic fraction concentrated under reduced pressure to afford the required dark green liquid compound and further color removed through charcoal decolorization on filteration then dissolved in methanol and poured into diethyl ether to afford white solid compound it is used for next step without further purification. In one embodiment the organic amine base used is a triethylamine. The reaction mixture is diluted with Ethyl acetate washed successively with water, dried with sodium sulphate (Na₂So₄), and then filtered and the filtrate is evaporated to dryness. The filtrate is diluted with methanol and poured into diethyl ether to solidify the compound 8.

Step-(g): Anomeric benzylation of 2-Azido-D-Glucosamine [Compound 9]

Step (g) involves anomeric benzylation of a compound 8 to obtain a compound of Formula 9 as schematically presented below:

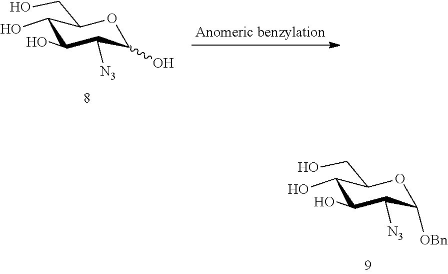

Anomeric benzylation takes place by treating the compound 8 with alcohol while stirring. The reaction may be carried out in the presence of Lewis acid at high temperature Preferably in one embodiment stirring is done at 60° C. and evaporation are done at 50° C. In another embodiment reaction carried out in benzyl alcohol as solvent and Lewis acid is boron trifluoride dietherate. The reaction mixture is poured into Et₂O and then stirred at 0° C. residue formed and filtered. Preferably the alcoholic solvent used is isopropanol. Further crystallization affords white crystalline solid. The solid obtained may be washed with an alcoholic solvent such as isopropanol and followed by diethyl ether.

Step-(h): Installing a 4,6-O-benzylidene protecting group [Compound 10].

Step (h) involves benzylidene protection of the compound of Formula 9 to obtain a compound of Formula 10 as schematically presented below;

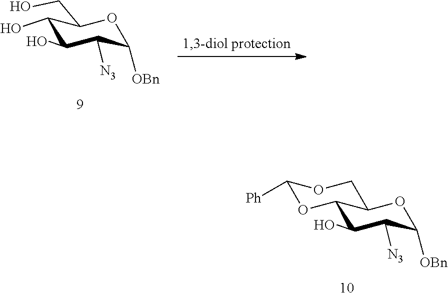

This benzylidene protection is performed by treating a compound with structural Formula-9 with dimethyl benzaldehyde acetal. The reaction can be performed in the presence of Organic acid. In one embodiment the Organic acid used is a p-toulene sulfonic acid.

The mixture stirred at room temperature for 4 to 8 hours, preferably for 6 hours and then stirred at rt, preferably dissolve a small amount of 5% EtOAc/n-Hexane followed by stirring at room temperature for 30 minutes at rt.

After proper stirring, the reaction residue filtered on Buchner funnel under continuous suction followed by washing with 5% EtOAc/n-Hexane of solvents, and water. The precipitate washed with water once under continuous suction until getting free white powder. The white powder is dissolved with EtOAc and slowly added n-Hexane until precipitate out filtered on a filter paper. After drying Benzyl 2-azido-4, 6-O-benzylidene-2-deoxy-D-glucopyranoside [Compound 10] is obtained. The drying can be carried out in vacuum at room temperature over phosphorous pentoxide (P₂O₅)

Step-(i): O-Alkylation [Compound 11].

Step-(i) involves O-alkylation of the compound of Formula 10 by treating with (S)-(2)-chloropropionic acid in presence of an appropriate solvent to obtain a compound 11

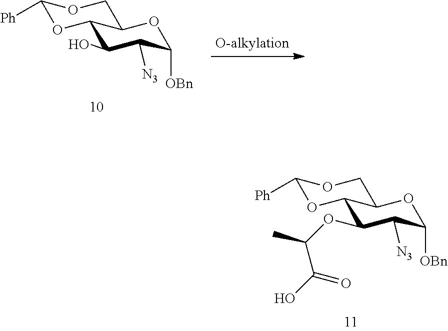

The solvent in O-Alkylation step (i) is dry DMF. Any other similar inert high boiling solvents can also be used. The reaction can be performed in the presence of metal hydride such as sodium hydride, potassium hydride, calcium hydride etc. In one preferred embodiment, the metal hydride used in step-(i) is sodium hydride. Initially metal hydride is added at 0° C. in dry DMF under stirring for 10 minutes compound IV added slowly then after 30 min (S)-(2)-chloropropionic acid in dry DMF more diluted was added slowely through dropping funnel. After completion of reaction, the reaction mixture poured into crushed ice and adjusted ph 2-3 until forms precipitate then filtered it out. The solid compound recrystalized in 10% MeOH/DCM to give O-alkylated monosaccharide Compound 11 is obtained as white solid.

Step-(j): Peptide Coupling [Compound 12].

Step-(j) involves the coupling of peptides by treating the compound of Formula 11 with L-alanyl-D-isoglutamine benzyl ester trifluoroacetate to obtain a compound Formula 12 as schematically presented below;

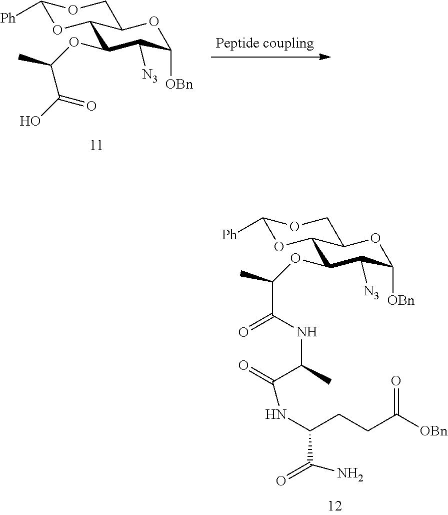

The coupling takes place under standard carbodiimide coupling conditions such as in the presence of 1-Ethyl-3-(-3-dimethyl aminopropyl) carbodiimide (EDCI) and Hydroxybenzotriazol (HOBt).Step-(j) can be performed in the presence of a base. In one embodiment the base is N.N-Diisopropylethylamine (DIPEA). The reaction mixture is stirred at RT for 12 to 18 hours, preferably 15 hours. After concentrating, the residue can be extracted with an organic solvent such as chloroform and followed by washing the organic layer with sat bicarbonate solution, dried and evaporated. The residue obtained can be re-crystalized in alcohol/DCM solvent such as DCM, alcohols such as methanol, ethanol etc. or mixture thereof. In one embodiment 5% DCM/Methanol mixture is used for re-crystallization.

Step-(k): Azide reduction to amine [Compound 13].

Transformation of amine from azide by using compound of Formula 12 with triphenylphoshine in a suitable solvent system to obtain a compound of Formula 13 as schematically presented below;

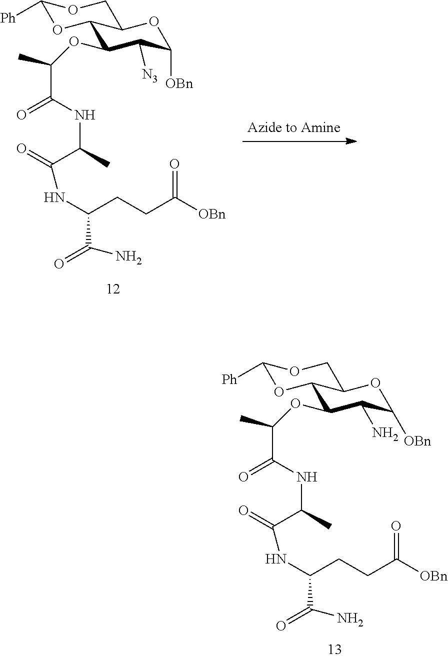

The Transformation of amine from azide carried out in presence of trialkylphoshine in THF/H₂O in the portion of (3:1) then Transformation of amine from azide The reaction mixture is stirred at RT for 12 to 18 hours, preferably 16 hours. After concentrating, the residue can be dissolved in DCM and added Diethylether to afford compound VII; Step-(k) can be performed in the presence of Phosphine ligand.

In one embodiment the phoshine ligand can be triphenylphosphine (PPh₃)

In one embodiment the reducing agents can be THF/H₂O in the portion of (3:1)

Step-(l): general procedure for acid amine coupling

Step-(l) involves the coupling of alkyl or alicyclic carboxylic acid by treating the compound of Formula 13 with suitable coupling agent to obtain a compound of Formula 14 as schematically presented below;

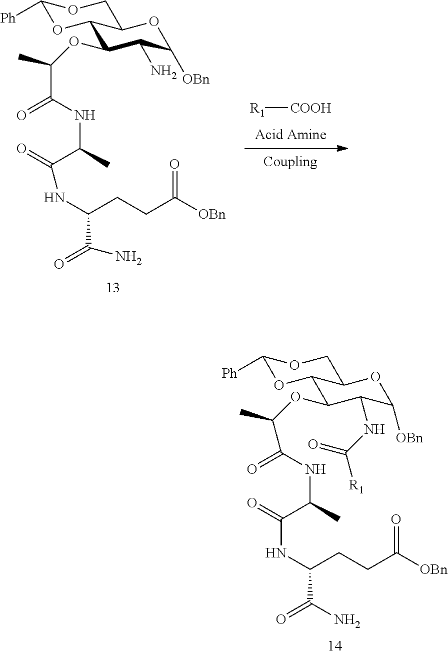

wherein R₁ is selected from substituted or unsubstituted (C₃-C₆) cycloalkyl, 3 to 6 membered heterocycloalkyl with one or more hetero atoms selected from Nitrogen and Oxygen or

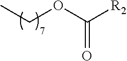

wherein R₂ is selected from group consisting of C₁ to C₁₈ aliphatic chain.

The coupling takes place under standard carbodiimide coupling conditions such as in the presence of 1-Propanephosphonic acid anhydride (T3P). Step-(1) can be performed in the presence of a base. In one embodiment the base is N.N-Diisopropylethylamine (DIPEA). The reaction mixture is stirred at RT for 12 to 18 hours, preferably 15 hours. After concentrating, the residue can be extracted with an organic solvent such as chloroform and followed by washing the organic layer with sat bicarbonate solution, dried and evaporated. The residue obtained can be recrystalized in solvent such as chloroform, alcohols such as methanol, ethanol etc. or mixture thereof. In one embodiment chloroform-methanol mixture is used for recrystalization.

Step-(m): Removal of protection to obtain structural formula-I

Step-(m) involves hydrogenolysis De-protection is carried out by treating the compound of Formula 14 with acetic acid, water and EtOAc in suitable metal catalyst for removal of protecting groups to obtain the desired compound as represented by ##structural Formula-I ##.

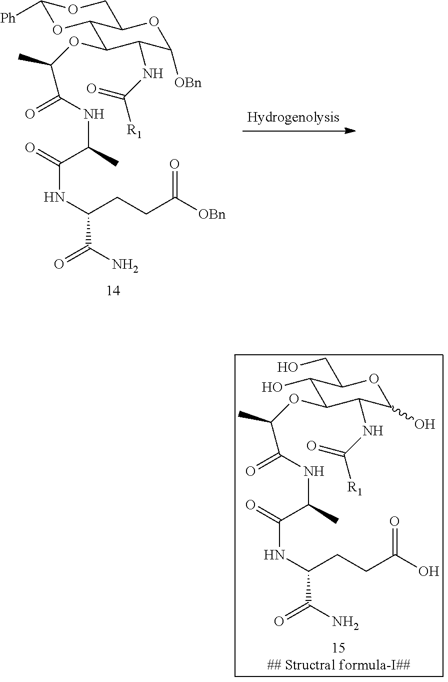

wherein R₁ is selected from substituted or unsubstituted (C₃-C₆) cycloalkyl, 3 to 6 membered heterocycloalkyl with one or more hetero atoms selected from Nitrogen and Oxygen or

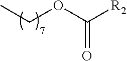

wherein R₂ is selected from group consisting of C₁ to C₁₈ aliphatic chain.

In one embodiment the acid used in step-(m) for deprotection is glacial acetic acid. The de-protection can be performed in the presence of a catalyst. In one embodiment the catalyst used in step-(m) is palladium black. To a solution of compound Formula-14 dissolved in acetic acid added with catalyst. And the compound subjected to hydrogenolysis for 12 h to 48 h.

The catalyst is filtered off, and, after addition of water, the filtrate is evaporated under diminished pressure by azeotroping with toluene. The residue can be dissolved in water and lyophilized. In one embodiment the residue is dissolved in a small volume of water and then applied to a column of Sephadex LH-20. The purified fractions can be lyophilized.

In a preferred embodiment of the present invention the following compounds of formula I are prepared wherein R₁ is selected from 15b to 15i.

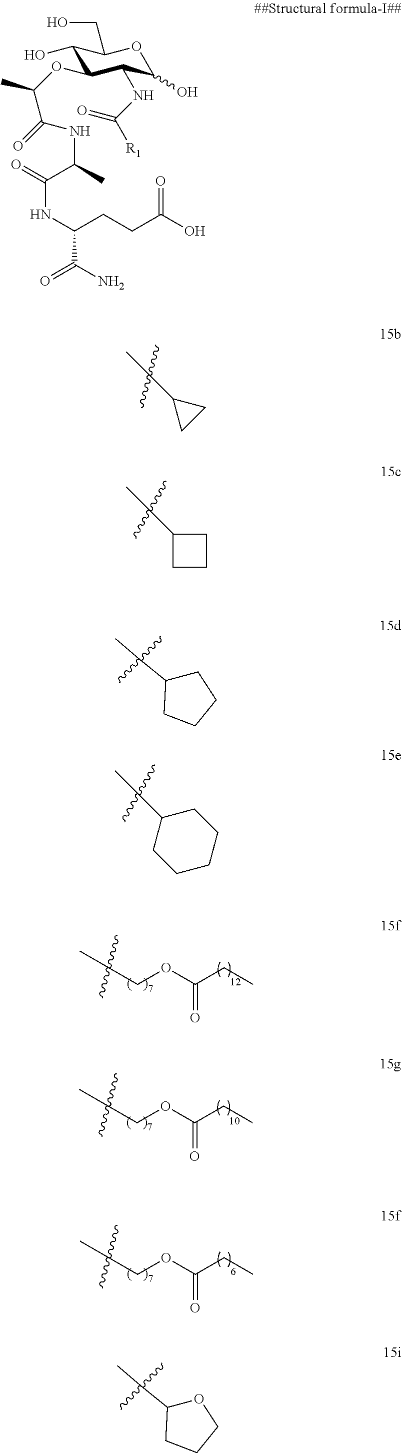

### EXAMPLES

The Muramyl Dipeptide compound its intermediates, the process for their synthesis and evaluation of these Muramyl Dipeptide compounds as an adjuvant are further explained and demonstrated by way of below non-limiting examples.

The reaction steps shown in above FIG-1 are further described in below experimental procedure step-a to step-f.

### Example 1

The synthesis of the compound of formula I involves the following steps:

### FIG. 1: Experimental Procedure for the Synthesis of L-Alanyl-D-Isoglutamine Benzyl Ester:

The novel Muramyl Dipeptide derivatives as described and disclosed in this invention contains an L-alanyl-D-isoglutamine dipeptide entity. The L-alanyl-D-isoglutamine benzyl ester required for the final preparation of novel Muramyl Dipeptide derivatives according to the present invention was synthesized by the method as shown in above reaction FIG. 1 and as described below

### Step-a): Preparation of (R)-2-amino-5-(benzyloxy)-5-oxopentanoic acid [Compound 2]

A mixture of D-Glutamic acid [Compound 1](4.0 g, 27.2 mmols) and anhydrous. Na₂SO₄ (4.0 g) was suspended in benzyl alcohol (50 mL, 484 mmol) and BF₃. Et₂O (54%, 7.4 ml, 54.4 mmols) was added by means of a syringe. The suspension was stirred at RT for 14 hrs. The mixture was diluted with absolute THF (150 mL) and filtered with the aid of charcoal. The clear filtrate was treated with Et₃N (8.2 mL, 59.2 mmols) and concentrated under vacuum until a slurry residue was formed.

The viscous residue was triturated with EtOAc (200 mL) and the precipitated solid was isolated by suction and washed with additional solvent to afford [compound 2] as a white solid (6.04 g, yield 93%, with respect to the starting material).

### Step-b): Preparation of (R)-5-(benzyloxy)-2-(tert-butoxy carbonyl amino)-5-oxopentanoic acid [Compound 3]

To a solution of the [compound 2](6.0 g, 25.3 mmol) in dioxane and water (1:1, 40 mL) at 0° C. was added Boc₂O (6.62 g, 30.36 mmol) and the mixture was stirred overnight (15 hours). The solvent was removed under reduced pressure and the residue was diluted with water (30 mL), basified with Na₂CO₃ and washed with EtOAc (3×100 mL). The aqueous layer was adjusted to pH 2-3 with a 5M aqueous HCl solution and extracted with EtOAc (4×100 mL). The combined organic extracts were washed with brine, dried over Na₂SO₄ and the solvent was removed under reduced pressure to afford (R)-5-(benzyloxy)-2-(tert-butoxycarbonyl)-5-oxopentanoic acid [compound 3](8.19 g, 95%) as a viscous colourless oil. with the following NMR characteristics. 1H NMR (500 MHz, CDCl3) δ 7.42-7.25 (m, 5H), 5.25 (br, 1H), 5.12 (s, 2H), 4.32 (t, J=24.5 Hz, 1H), 2.57-2.42 (m, 2H), 2.29-2.19 (m, 1H), 2.02 (dd, 1H), 1.44 (s, 9H) ppm; ESI-MASS m/z: m/z Calculated 237, found 260 [M+Na]+.

### Step-c): Preparation of (R)-benzyl 5-amino-4-((tert-butoxy carbonyl) amino)-5-oxopentanoate [Compound 4]

To a solution of above acid [Compound 3](7.0 g, 20.8 mmol) in THF (15 mL) were added ethyl chloroformate (2.7 mL, 28.36 mmol) and triethylamine (4.21 mL, 30.25 mmol) at 0° C. The reaction mixture was stirred at 0° C. for 0.5 hrs, then cooled to −15° C. followed by addition of a methanolic solution of ammonia (25 mL, 4.0 M) was added. Then the reaction mixture was stirred at −15° C. for another 1.5 hrs and diluted with ethyl acetate (200 mL). The organic phase was washed with water (100 mL×3) and then followed by washing with brine solution (100 mL) and dried over anhydrous sodium sulphate. The solvent was removed in vacuum and the residue was purified by flash chromatography to furnish the desired [compound 4](6.63 g, 90%) as a white solid. characterized by NMR. 1H NMR (400 MHz, CDCl3) δ 7.39-7.29 (m, 5H), 6.35 (br, 1H), 5.72 (br, 1H), 5.11 (s, 2H), 3.7 (s, 2H), 2.62-2.40 (m, 2H), 2.31-1.94 (m, 2H), 1.43 (s, 9H) ppm; ESI-MASS m/z: Calculated 236, found 237 [M+H]+.

### Step-d): (R)-benzyl 5-amino-4-((S)-2-((tert-butoxy carbonyl)amino) propanamido)-5-oxopentanoate [Compound 5]

t-Butoxycarbonyl-D-isoglutamine benzyl ester [Compound 4](6.5 g, 19.3 mmols) was dissolved in cold trifluoroacetic acid (4 mL in 16 mL DCM) and the resulting solution was stirred at room temperature for 15 mins. Trifluoroacetic acid was then removed and the residue was triturated with Et₂O. This D-isoglutamine benzyl ester trifluoroacetate was dried over on KOH pellets followed by using trap.

To t-butoxycarbonyl-L-alanine (4.012 g, 21.23 mmols) in dry THF, EDCI (4.46 g, 23.36 mmols) and HOBt (3.57 g, 23.36 mmols). were added at 0° C. After stirring at RT for 30 mins, to this solution D-isoglutamine benzyl ester trifluoroacetate (dissolved in THF) was added followed by DIPEA (7.06 ml, 40.53 mmols). The reaction was stirred at RT for 15 hrs, the solution was then concentrated and the residue extracted with EtOAc (500 ml). The EtOAc layer was washed successively with 5% NaHCO₃, 10% citric acid, and water (200 ml×3), then dried over Na₂SO₄ and evaporated. The residue was triturated with petroleum ether giving crystals which were recrystallized from EtOAc-petroleum ether to yield [compound 5](6.37 g, yield 81%) as a white solid with the following NMR Characterization.

1H NMR (400 MHz, CDCl3): δ 7.39-7.30 (m, 5H), 6.81 (br, 1H), 5.78 (br, 1H), 5.20 (d, J=3.2 Hz, 1H), 5.12 (ABq, J=12.3 Hz, 2H), 4.49 (dd, J=10.1, 7.6 Hz, 1H), 4.07 (dq, J=7.2, 3.2 Hz, 1H), 2.56 (dt, J=16.9, 7.2 Hz, 1H), 2.22 (m, 1H), 2.48 (dt, J=17.0, 6.7 Hz, 1H), 2.26-2.19, (m, 1H), 2.05-1.98 (m, 1H), 1.41 (s, 9H), 1.32 (d, J=7.1 Hz, 3H) ppm; ESI-MASS m/z: Calculated 407, found [M+Na]+430

Step-e): benzyl (R)-5-amino-4-((S)-2-aminopropanamido)-5-oxopentanoate trifluoroacetate [Compound 6](t-Butoxycarbonyl-L-alanyl-D-isoglutamine benzyl ester (25 g, 84.2 mmols) was dissolved in cold trifluoroacetic acid (20 mL) and DCM (80 mL) the resulting solution was stirred at room temperature for 30 mins. Trifluoroacetic acid was then removed by making azeotropic mixture with touelene and the residue was triturated with Et₂O to obtain L-alanyl-D-isoglutamine benzyl ester trifluoroacetate [compound 6]. This L-alanyl-D-isoglutamine benzyl ester trifluoroacetate was dried over KOH pellets followed by using a trap. To afford oily compound 6 following NMR Characterization.

### FIG. 2:

The synthesized peptide fragment of compound 6 as shown in example-1 used to couple with Sugar fragment of compound 11 which has shown in FIG. 2

### Step-f): Preparation of (3R,4R,5S,6R)-3-azido-6-(hydroxymethyl)tetrahydro-2H-pyran-2,4,5-triol (8)

A typical experimental procedure for the preparation of triflyl azide was as following: To the mechanically stirred suspension of sodium azide (43.6 g, 67.0 mmol) in 600 mL of acetonitrile was cooled in ice bath. Then triflic anhydride (157 g, 55.6 mmol) was added to the mixture through addition funnel during 45 min while stirring. After the reaction was maintained for 2 h in ice bath, the TfN₃-containing solution (filtration of the salts can be done if necessary) was added directly to the mechanically stirring solution of amine solution for the subsequent diazotransfer reaction.

General procedure for the reaction of triflyl azide with amines. For organic soluble substrates, 100 g of [Compound 7] was dissolved in 250 mL of acetonitrile. In case of saline substrates, water was employed instead. Then 1% equiv of CuSO₄ and 2 equiv of NEt₃ per substrate amine were added into the solution while stirring. The mixture was cooled in an ice bath for a while, acetonitrile solution of triflyl azide (1.2 equiv per amino group, based on the amount of triflic anhydride used in the preparation of TfN₃) was then added into the mixture dropwise. The reaction mixture was allowed to warm to room temperature. Generally speaking, a homogeneous solution could be obtained after the addition of triflyl azide, and the reaction normally went to completion within 12 h. The solvent was removed under reduced pressure. The residue was Recrystallized by dissolving in DCM then poured into n-hexane to afford (3R,4R,5S,6R)-3-azido-6-(hydroxymethyl)tetrahydro-2H-pyran-2,4,5-triol [Compound 8](108 g, 94%) as white solid characterized by 1H-NMR and MASS-ESI 1H NMR (500 MHz, CD3OD) 1H NMR (500 MHz, CDCl3) δ 5.06-5.01 (d, J=3.7 Hz, 1H, anomeric) 4.77-4.72 (d, J=12.0 Hz, 1H), 4.62 (d, J=12.0 Hz, 1H), 4.32-4.20 (m, 2H), 3.91 (m, 1H), 3.74-3.71 (t, J=10.4 Hz, 1H), 3.54-3.50 (t, J=9.3 Hz, 1H) ppm; ESI-MS: m/z Calcd. for C₆H₁₁N₃O₅ 205 [M+Na]+: 228:

### Step-g): Preparation of (2R,3S,4R,5R,6S)-5-azido-6-(benzyloxy)-2-(hydroxymethyl)tetrahydro-2H-pyran-3,4-diol [Compound 9]

### Anomeric benzylation of 2-azido D-Glucosamine (8)

To the mechanically stirred suspension of 2-azido D-glucosamine [compound 8](85 g, 0.39 mmol) and Acetyl chloride (27.4 mL, 0.39 mmol) was slowly added to a in anhydrous benzyl alcohol (200 mL) under nitrogen atmosphere. The suspension was stirred at room temperature for 2 h, heated to 50° C. for 8h, and then cooled to room temperature. The resulting yellow solution was slowly poured onto Et₂O (3L) in ice-water bath, and the mixture was vigorously stirred for 2 h at 0° C. The precipitate was recovered by filtration and dried under vacuum to afford (2R, 3S,4R,5R,6S)-5-azido-6-(benzyloxy)-2-(hydroxymethyl)tetrahydro-2H-pyran-3,4-diol [Compound 9](118 g, 96%) as a white solid characterized by 1H-NMR and MASS-ESI 1H NMR (500 MHz, CD3OD) 1H NMR (500 MHz, CDCl3) δ 7.51-7.46 (m, 2H), 7.43-7.31 (m, 3H), 5.00-4.95 (d, J=3.7 Hz, 1H, anomeric), 4.77-4.73 (d, J=12.0 Hz, 1H), 4.62 (d, J=12.0 Hz, 1H), 4.32-4.20 (m, 2H), 3.91 (m, 1H), 3.74 (t, J=10.4 Hz, 1H), 3.54 (t, J=9.3 Hz, 1H), 3.31-3.29 (dd, J=10.0, 3.7 Hz, 1H) ppm; ESI-MASS m/z: m/z Calculated. C₁₃H₁₇N₃O₅ 295 [M+Na]+318 [M+K]+; found: 334.12

### Step-h): Preparation of (2R,4aR,6S,7R,8R,8aS)-7-azido-6-(benzyloxy)-2-phenylhexahydropyrano[3,2-d][1,3]dioxin-8-ol [Compound 10]

### Benzylidene protection of compound (9)

(2R,3S,4R,5R,6S)-5-azido-6-(benzyloxy)-2-(hydroxymethyl)tetrahydro-2H-pyran-3,4-diol [Compound 9](80 g, 0.271 mol) was added to a mechanically stirred mixture of dimethyl benzaldehyde acetal (16.5 ml, 1.35 mol in dry ACN). and PTSA (10 g, 0.27 mol) were added then This mixture was stirred at room temperature for 2 hours, then stirred at rt for 2 hours to dissolve a small amount of remaining solid, then stirred at room temperature for 6 hours. The well-stirred reaction solution was now diluted with petroleum ether (300 ml), absolute ethanol (EtOH) for an amount of 100 ml) and 45 ml of water (H₂O). The reaction mixture was stirred for 1 day at room temperature, then stored at 0° C. for 2 days. The curdy white precipitate so formed was collected on a coarse glass frit funnel, drained thoroughly, then washed by re-suspension in absolute EtOH (approx. 100 ml). The white finely divided solid was drained thoroughly, re-suspended in diethyl ether (approx. 500 ml), re-drained thoroughly, and then dried in vacuum at room temperature over phosphorus pentoxide (P₂O₅) to get (2R,4aR,6S,7R,8R,8aS)-7-azido-6-(benzyloxy)-2-phenylhexahydropyrano[3,2-d][1,3]dioxin-8-ol [Compound 10](99 g, yield 95%) as a white solid characterized by 1H-NMR and MASS-ESI 1H NMR (500 MHz, CDCl3) 1H NMR (500 MHz, CDCl3) δ 7.51-7.46 (m, 2H), 7.43-7.31 (m, 8H), 5.54 (s, 1H), 5.00 (d, J=3.7 Hz, 1H, anomeric), 4.77 (d, J=12.0 Hz, 1H), 4.62 (d, J=12.0 Hz, 1H), 4.32-4.20 (m, 2H), 3.91 (m, 1H), 3.74 (t, J=10.4 Hz, 1H), 3.54 (t, J=9.3 Hz, 1H), 3.31 (dd, J=10.0, 3.7 Hz, 1H), 2.69 (s, 1H) ppm; ESI-MS: m/z Calcd. for C₂₀H₂₁N₃O₅[M 30+H]+: 384.

### Step-i): preparation of (R)-2-(((2R,4aR,6S,7R,8R,8aS)-7-azido-6-(benzyloxy)-2-phenylhexahydropyrano[3,2-d][1,3]dioxin-8-yl)oxy)propanoic acid [Compound 11]

### O-Alkylation: of compound (10)

To the mechanically stirred solution of Sodium hydride 60% dispersion in mineral oil, (50 g, 0.130 mol) was washed with hexanes (50 mL) three time to remove mineral oil and was suspended in 1Ltr anhydrous DMF under argon. [Compound 10](35 g, 1.56 mol) was added into the above solution slowly, followed by (S)-2-chloropropanoic acid (2.2 mL, 20.5 mmol) added dropwise over 30 min through addition funnel in 150 mL anhydrous DMF The resulting mixture was stirred vigorously at room temperature until there was no gas evolution. Then it was stirred at rt for 14 h stirring). The reaction mixture was cooled down to room temperature, after completion of reaction quenched with 200 mL of DI water and acidified with 1N HCl to pH=3. It forms precipitate then filtered and washed with 30% ethyl acetate/n-Hexane (2×1Ltr), and dried under continuous vacuum until dry to afforded product solid (R)-2-(((2R,4aR,6S,7R,8R,8aS)-7-azido-6-(benzyloxy)-2-phenylhexahydropyrano[3,2-d][1,3]dioxin-8-yl)oxy)propanoic acid [Compound 11](54 g, 91%) as an off white solid characterized by 1H-NMR and MASS-ESI; ¹H NMR (500 MHz, CDCl3): δ 7.50-7.41 (m, 2H), 7.41-7.31 (m, 8H), 5.56 (s, 1H), 5.06 (d, J=3.7 Hz, 1H, anomeric), 4.75 (d, J=11.9 Hz, 1H), 4.62 (d, J=11.9 Hz, 1H), 4.50 (q, J=13.9, 6.9 Hz, 1H), 4.23 (dd, J=10.3, 4.8 Hz, 1H), 4.05 (t, J=9.5 Hz, 1H), 3.90 (m, 1H), 3.74 (t, J=10.3 Hz, 1H), 3.64 (t, J=9.3 Hz, 1H), 3.42-3.38 (m, 1H), 1.47 (d, J=6.9 Hz, 3H) ppm; ESI-MASS: m/z Calcd. for C₂₃H₂₅N₃O₇ [M+H]+: 456.

### Step-j): (R)-benzyl-5-amino-4-((S)-2-((R)-2-(((2R,4aR,6S,7R,8R,8aS)-7-azido-6-(benzyloxy)-2-phenylhexahydropyrano[3,2-d][1,3]dioxin-8-yl)oxy)propanamido)propanamido)-5-oxopentanoate [compound 12]

### Peptide Coupling of Azido Muramic Acid (11)

Compound 6 (25 g) dissolved in THF (100 mL) with DIPEA (22 mL 123 mmol) was treated with Compound 11 (30 g, 61.9 mmol) in dry THF (500 mL) and EDC-HCl (1.27 g, 92.9 mmol). A catalytic amount of HOBt was added to the solution. The solution was stirred 8 h at room temperature. After the complete consumption of the starting material, the solvent was removed and the residue was extracted with EtOAc (500 mL) and washed with 1N HCl (250 mL×2) and saturated NaHCO₃ (250 mL×2) then brine (500 mL×2). The extract was dried over Na₂SO₄, concentrated, filtered, and recrystalized in 5% DCM/Methanol into give (R)-benzyl-5-amino-4-((S)-2-((R)-2-(((2R,4aR,6S,7R,8R,8aS)-7-azido-6-(benzyloxy)-2-phenylhexahydropyrano[3,2-d][1,3]dioxin-8-yl)oxy)propanamido)propanamido)-5-oxopentanoate [Compound 12](38 g, 93%) as a white solid. ¹H NMR (400 MHz, CDCl3): δ 7.65 (t, J=8.8 Hz, 1H), 7.46-7.40 (m, 2H), 7.40-7.30 (m, 12H), 7.15 (d, J=8.0 Hz, 1H), 6.79 (s, 1H), 5.55 (s, 1H), 5.45 (s, 1H), 5.04 (d, J=2.4 Hz, 2H, anomeric), 4.76 (d, J=10.0 Hz, 1H), 4.62 (d, J=11.7 Hz, 1H), 4.46 (td, J=8.3, 4.6 Hz, 1H), 4.31-4.19 (m, 3H), 3.96-3.85 (m, 2H), 3.75 (t, J=10.3 Hz, 1H), 3.61 (t, J=9.3 Hz, 1H), 3.41 (dd, J=10.1, 3.7 Hz, 1H), 2.58 (m, 1H), 2.45 (m, 1H), 2.27-2.16 (m, 1H), 2.02 (m, 1H), 1.39 (d, J=7.0 Hz, 3H), 1.35 (d, J=6.7 Hz, 3H) ppm; ESI-MASS: m/z Calcd. for C₃₈H₄₄N₆O₁₀ [M+H]⁺: 745.

Step-k):(R)-benzyl-5-amino-4-((S)-2-((R)-2-(((2R,4aR,6S,7R,8R,8aS)-7-amino-6-(benzyloxy)-2-phenylhexahydropyrano[3,2-d][1,3]dioxin-8-yl)oxy)propanamido)propanamido)-5-oxopetanoate (13): To the mechanically stirred solution of [compound 12](20 g, 9.41 mmol) in tetrahydrofuran (200 mL), was added triphenylphosphine (8.4 g, 32.35 mmol) and water (70 mL). The reaction mixture was stirred at 60° C. for 16 hours. After the completetion of the starting material, solvent was removed and the residue was extracted with CH₂Cl₂ (2×100 mL) and brine (50 mL×2). The extract was dried over MgSO₄, concentrated, and disolved in DCM then diethyl ether were added till precipitate out the compound followed by decanting excess triphenylphoshine decanted which disolved in diethyl ether and smae repeated once to give white solid, benzyl (R)-4-((S)-2-((R)-2-(((2R,4aR,6S,7R,8R,8aS)-7-alkaamido-6-(benzyloxy)-2-phenylhexahydropyrano[3,2-d][1,3]dioxin-8-yl)oxy)propanamido)propanamido)-5-amino-5-oxopentanoate to afford [Compound 13](17.8 g, 92%) as a white solid.characterized by ¹HNMR, MASS-ESI ¹H NMR (500 MHz, CDCl₃): δ7.39-7.33 (m, 2H), 7.34-7.23 (m, 15H), 7.04 (d, J=8.1 Hz, 1H), 6.74 (s, 1H), 5.50 (s, 2H), 5.23 (s, 1H), 5.02 (d, J=7.2 Hz, 2H), 4.78 (d, J=4.0 Hz, 1H), 4.68 (d, J=11.7 Hz, 2H), 4.44 (d, J=11.7 Hz, 1H), 4.39 (m, 1H), 4.23 (m, 1H), 4.17 (m, 1H), 3.76 (m, 1H), 3.67 (t, J=10.2 Hz, 1H), 3.59-3.50 (m, 3H), 2.49 (m, 1H), 2.39 (m, 1H), 2.14 (m, 1H), 1.95 (m, 1H), 1.34 (d, J=7.0 Hz, 3H), 1.29 (d, J=7.0 Hz, 3H) ppm; ESI-MASS: m/z Calcd. for C₃₈H₄₆N₄O₁₀ 718 [M+H]⁺: 719.

The Synthesized Compound 13 as Shown in Example-1 (FIG. 2) Used to Coupled with Cycloalkyl Carboxylic Acid or Mixed Ester Carboxylic Acid to Obtain Compounds (14a to 14h) which as Described in Step-l Given Below

### Step-1): General Procedure for Acid Amine Coupling

A mechanically stirred solution of cyclo alkyl carboxylic acid or mixed ester carboxylic acid (13 g) in anhydrous tetrahydrofuran (100 mL) T3P (7.5 mL, 25 mmol and DIPEA (9 mL, 50.4 mmol) were added and stirred 30 min at 0° C. and compound 13 (12 g) in (50 mL) anhydrous THF were added through addition funnel to the stirring reaction mixture then keep it stirring at RT for 14 h. After the consumption of the starting material, solvent was removed and the residue was extracted with EtOAc (150 mL) and washed with 1N HCl (20 mL×2) then brine (wash 50 mL×2). The extract was dried over MgSO₄, concentrated, and recrystalized in (5% MeOH in DCM) to give benzyl(R)-4-((S)-2-((R)-2-(((2R,4aR,6S,7R,8R,8aS)-7-alkaamido-6-(benzyloxy)-2-phenylhexahydropyrano[3,2-d][1,3]dioxin-8-yl)oxy)propanamido)propanamido)-5-amino-5-oxopentanoate to afford (14a to 14h) in the ranges of the yields (90-95%) as white solid compounds.

i).benzyl(R)-4-((S)-2-((R)-2-(((2R,4aR,6S,7R,8R,8aS)-7-acetamido-6-(benzyloxy)-2-phenylhexahydropyrano[3,2-d][1,3]dioxin-8-yl)oxy)propanamido)propanamido)-5-amino-5-oxopentanoate (14a 11.8 g, 92%) as a white solid characterized by ¹HNMR, MASS-ESI ¹H NMR (300 MHz, CDCl3):δ 7.45-7.37 (m, 2H), 7.34-7.22 (m, 13H), 5.52 (s, 1H), 5.04 (s, 2H), 4.94-4.87 (d, J=3.7 Hz, 1H), 4.49-4.32 (dd, J=57.8, 11.9 Hz, 1H), 4.30 (dd, J=8.9, 4.7 Hz, 1H), 4.22-4.03 (m, 3H), 3.83-3.53 (m, 3H), 3.29 (s, 1H), 2.34-2.31 (s, 3H), 2.29-2.21 (m, 2H), 2.12 (dt, J=12.4, 7.6 Hz, 1H), 1.84 (dt, J=14.2, 8.4 Hz, 1H), 1.41 (td, J=7.8, 4.0 Hz, 1H), 1.39-1.27 (dd, 6H) ppm; ESI-MASS: m/z Calcd. for C₄₀H₄₈N₄O₁₁ 760.33 found [M+Na]⁺: 783.

ii). 2.7.2: benzyl (4R)-5-amino-4-((2S)-2-((2R)-2-(((2R,4aR,7R,8R,8aS)-(benzyloxy)-7-(cyclopropanecarboxamido)-2-phenylhexahydropyrano[3,2-d][1,3]dioxin-8-yl)oxy)propanamido)propanamido)-5-oxopentanoate (14b, 12.4 g, 94%) as a white solid characterized by 1H NMR, MASS-ESI, 1H NMR (300 MHz, CDCl3) δ 7.48-7.37 (m, 2H), 7.36-7.20 (m, 13H), 5.56-5.50 (s, 1H), 5.10-5.02 (s, 2H), 4.98-4.93 (d, J=3.7 Hz, 1H), 4.72-4.62 (d, J=11.9 Hz, 1H), 4.52-4.44 (d, J=11.9 Hz, 1H), 4.36-4.27 (dd, J=8.9, 4.7 Hz, 1H), 4.24-4.06 (m, 3H), 3.86-3.52 (m, 5H), 2.45-2.28 (m, 2H), 2.24-2.06 (m, 1H), 1.96-1.75 (m, 1H), 1.50-1.37 (m, 1H), 1.35-1.20 (dd, J=10.9, 9.7 Hz, 6H), 0.87-0.78 (m, 2H), 0.75-0.62 (m, 2H) ppm; ESI-MASS: m/z Calcd. for C₃₈H₄₆N₄O₁₁ 786. found [M+H]⁺ 787, [M+Na]⁺809

iii). benzyl (4R)-5-amino-4-((2S)-2-((2R)-2-(((2R,4aR,7R,8R,8aS)-6-(benzyloxy)-7-(cyclobutanecarboxamido)-2-phenylhexahydropyrano[3,2-d][1,3]dioxin-8-yl)oxy)propanamido)propanamido)-5-oxopentanoate (14c, 12.5 g, 93%) as a white solid characterized by 1HNMR, MASS-ESI, 1H NMR (300 MHz, CDCl3) δ 7.50-7.38 (m, 2H), 7.35-7.21 (m, 13H), 5.55-5.49 (s, 1H), 5.08-5.03 (s, 2H), 4.99-4.94 (d, J=3.7 Hz, 1H), 4.72-4.61 (d, J=11.9 Hz, 1H), 4.51-4.43 (d, J=11.9 Hz, 1H), 4.37-4.28 (dd, J=8.9, 4.7 Hz, 1H), 4.25-4.05 (m, 3H), 3.87-3.51 (m, 5H), 3.20-3.15 (m, 1H), 2.46-2.27 (m, 2H), 2.25-2.07 (m, 1H), 1.95-1.74 (m, 1H), 1.42-1.25 (m, 10H), 0.92-0.77 (m, 2H) ppm; ESI-MASS: m/z Calcd. for. C₄₃H₅₂N₄O₁₁, 800.36 found [M+H]⁺ 801., [M+Na]⁺824

iv). benzyl (4R)-5-amino-4-((2S)-2-((2R)-2-(((2R,4aR,7R,8R,8aS)-6-(benzyloxy)-7-(cyclopentanecarboxamido)-2-phenylhexahydropyrano[3,2-d][1,3]dioxin-8-yl)oxy)propanamido)propanamido)-5-oxopentanoate (14d, 13 g, 95%) as a white solid characterized by 1HNMR, MASS-ESI, 1H NMR (400 MHz, CDCl3) δ 7.52-7.36 (m, 4H), 7.34-7.19 (m, 11H), 5.54-5.49 (s, 1H), 5.09-5.02 (s, 2H), 4.98-4.95 (d, J=3.7 Hz, 1H), 4.73-4.60 (d, J=11.9 Hz, 1H), 4.54-4.42 (d, J=11.9 Hz, 1H), 4.36-4.27 (dd, J=8.9, 4.7 Hz, 1H), 4.27-4.04 (m, 3H), 3.88-3.50 (m, 5H), 2.47-2.26 (m, 3H), 2.27-2.06 (m, 1H), 1.94-1.75 (m, 1H), 1.45-1.27 (m, 10H), 1.15-0.79 (m, 4H) ppm; ESI-MASS: m/z Calcd. for. C₄₄H₅₄N₄O₁₁, 814.38, found [M+H]⁺ 815,[M+K]⁺854.

v). benzyl (4R)-5-amino-4-((2S)-2-((2R)-2-(((2R,4aR,7R,8R,8aS)-6-(benzyloxy)-7-(cyclohexanecarboxamido)-2-phenylhexahydropyrano[3,2-d][1,3]dioxin-8-yl)oxy)propanamido)propanamido)-5-oxopentanoate (14e, 13.1 g, 94%) as a white solid characterized by 1H NMR (500 MHz, CDCl3) δ 7.51-7.37 (m, 4H), 7.34-7.19 (m, 11H), 5.53-5.48 (s, 1H), 5.08-5.01 (s, 2H), 4.97-4.95 (d, J=3.7 Hz, 1H), 4.72-4.61 (d, J=11.9 Hz, 1H), 4.53-4.42 (d, J=11.9 Hz, 1H), 4.35-4.28 (dd, J=8.9, 4.7 Hz, 1H), 4.25-4.03 (m, 4H), 3.89-3.49 (m, 4H), 2.46-2.25 (m, 3H), 2.27-2.06 (m, 1H), 1.94-1.79 (m, 1H), 1.74-1.51 (m, 4H), 1.47-1.29 (m, 10H), 1.18-0.90 (m, 2H) ppm; ESI-MASS: m/z Calcd. for. C₄₅H₅₆N₄O₁₁, 828.39, found [M+H]⁺ 829.20, [M+Na]⁺852

vi). 8-(((2R,4aR,7R,8R,8aS)-8-(((R)-1-(((S)-1-(((R)-1-amino-5-(benzyloxy)-1,5-dioxopentan-2-yl)amino)-1-oxopropan-2-yl)amino)-1-oxopropan-2-yl)oxy)-6-(benzyloxy)-2-phenylhexahydropyrano[3,2-d][1,3]dioxin-7-yl)amino)-8-oxooctyl tetradecanoate (14f, 16.5 g, 92%) as a white solid characterized by 1HNMR, MASS-ESI, 1H NMR (500 MHz, CDCl3): δ 7.86 (s, 1H), 7.45-7.40 (m, 3H), 7.37-7.27 (m, 12H), 7.13 (d, J=6.1 Hz, 1H), 6.90 (s, 1H), 6.29 (d, J=8.8 Hz, 1H), 5.54 (s, 1H), 5.08 (s, 2H), 4.93 (d, J=3.8 Hz, 1H), 4.71 (d, J=11.7 Hz, 1H), 4.51-4.43 (m, 2H), 4.30 (m, 1H), 4.23 (dd, J=10.2, 4.7 Hz, 1H), 4.15 (t, J=6.7 Hz, 1H), 4.04 (t, J=6.7 Hz, 2H), 3.86 (m, 1H), 3.76 (t, J=10.2 Hz, 1H), 3.67 (m, 1H), 2.56 (m, 1H), 2.45 (m, 1H), 2.27 (t, J=7.5 Hz, 2H), 2.21 (m, 1H), 2.16-2.08 (m, 2H), 2.02 (m, 1H), 1.62-1.57 (m, 3H), 1.53 (m, 1H), 1.38 (d, J=7.0 Hz, 3H), 1.34 (d, J=6.7 Hz, 3H), 1.30-1.23 (m, 28H), 0.88 (t, J=6.9 Hz, 3H) ppm; ESI-MASS: m/z Calcd. for. C₆₀H₈₆N₄O₁₃ 1070.61 found [M+H]⁺ 1071.

vii).8-(((2R,4aR,7R,8R,8aS)-8-(((R)-1-(((S)-1-(((R)-1-amino-5-(benzyloxy)-1,5-dioxopentan-2-yl)amino)-1-oxopropan-2-yl)amino)-1-oxopropan-2-yl)oxy)-6-(benzyloxy)-2-phenylhexahydropyrano[3,2-d][1,3]dioxin-7-yl)amino)-8-oxooctyl dodecanoate (14 g, 15.8 g, 90%) as a white solid characterized by 1HNMR, MASS-ESI, 1H NMR (500 MHz, CDCl3): δ 7.86 (s, 1H), 7.45-7.38 (m, 3H), 7.38-7.27 (m, 14H), 7.12 (d, J=6.1 Hz, 1H), 6.91 (s, 1H), 6.29 (d, J=8.8 Hz, 1H), 5.54 (s, 1H), 5.08 (s, 2H), 4.93 (d, J=3.8 Hz, 1H), 4.71 (d, J=11.7 Hz, 1H), 4.49 (d, J=11.7 Hz, 1H), 4.30 (m, 1H), 4.24 (dd, J=10.2, 4.7 Hz, 1H), 4.16 (t, J=6.7 Hz, 1H), 4.04 (t, J=6.7 Hz, 2H), 3.87 (m, 1H), 3.77 (t, J=10.2 Hz, 1H), 3.72-3.62 (m, 2H), 2.57 (m, 1H), 2.46 (m, 1H), 2.28 (t, J=7.5 Hz, 2H), 2.21 (m, 1H), 2.17-2.07 (m, 2H), 2.02 (m, 1H), 1.63-1.56 (m, 3H), 1.54-1.51 (m, 1H), 1.39-1.37 (d, J=7.0 Hz, 3H), 1.35-1.33 (d, J=6.7 Hz, 3H), 1.30-1.24 (m, 24H), 1.11-0.88 (t, J=6.9 Hz, 3H) ppm; ESI-MASS: m/z Calcd. for.C₅₈H₈₂N₄O₁₃ 1043 [M+H]⁺:1044

viii).8-(((2R,4aR,7R,8R,8aS)-8-(((R)-1-(((S)-1-(((R)-1-amino-5-(benzyloxy)-1,5-dioxopentan-2-yl)amino)-1-oxopropan-2-yl)amino)-1-oxopropan-2-yl)oxy)-6-(benzyloxy)-2-phenylhexahydropyrano[3,2-d][1,3]dioxin-7-yl)amino)-8-oxooctyl octanoate (14h, 15.3 g, 93%) as a white solid characterized bym HNMR, MASS-ESI, 1H NMR (500 MHz, CDCl3): δ 7.47-7.42 (m, 3H), 7.39-7.29 (m, 14H), 7.16 (d, J=7.9 Hz, 1H), 7.10 (d, J=6.0 Hz, 1H), 6.89 (s, 1H), 6.13 (d, J=9.2 Hz, 1H), 5.56 (s, 1H), 5.35 (s, 1H), 5.10-5.09 (s, 2H), 4.91 (d, J=3.8 Hz, 1H), 4.73 (d, J=11.7 Hz, 1H), 4.49 (d, J=11.6 Hz, 1H), 4.31 (m, 1H), 4.24 (dd, J=10.0, 4.5 Hz, 1H), 4.11 (m, 1H), 4.01 (m, 1H), 3.87 (m, 1H), 3.78 (d, J=10.1 Hz, 1H), 3.69-3.64 (m, 2H), 2.57 (m, 1H), 2.46 (m, 1H), 2.27-1.97 (m, 6H), 1.66 (s, 3H), 1.58-1.51 (m, 4H), 1.39 (d, J=7.1 Hz, 3H), 1.35 (d, J=6.7 Hz, 2H), 1.27-1.24 (m, 15H), 1.10-0.88 (t, J=6.6 Hz, 3H) ppm; ESI-MASS: m/z Calcd. for.C₅₄H₇₄N₄O₁₃ 986 [M+H]⁺: 987.

### The Synthesized Compound 14a to 14h as Shown in Example-1 (FIG. 2) Used to Hydrogenolysis to Obtain Compounds (15a to 15i) as Shown Below in Step-m

### Step-m): General Procedure for Hydrogenolysis

To a stirred solution of [compound 14](10 g, 1.65 mmol), THF (50 mL), acetic acid (20 mL) and water (100 mL) was added 10% Pd/C (3.5 g) added as catalyst. The resulting mixture was hydrogenated at room temperature by using a hydrogen-reacter for 16 h and filtered through a celite cake. After evaporation followed by azotropization then the residue was disolved in HPLC MeOH and diethyl ether were added untill precipitate stirred for 30 min to free of then ether decanted the process repeated once to afford compounds 15a to 15i in the ranges of yields (90-95%) as a white solid compounds

Example-2: The synthesis of the title compound (4R)-4-((2S)-2-((2R)-2-(((3R,4R,5S,6R)-3-acetamido-2,5-dihydroxy-6-(hydroxymethyl)tetrahydro-2H-pyran-4-yl)oxy)propanamido)propanamido)-5-amino-5-oxopentanoic acid (15a, 5.9 g, 92%) given as example 2 involves all the steps for the synthesis of peptide as given in FIG. 1 of example 1 and the specific sugar fragment prepared as explained in FIG. 2 of example 1 and their successive acid amine coupling and deprotection as given under example 1 and the final compound was obtained as a white solid characterized by ¹HNMR, MASS-ESI, ¹H NMR (400 MHz, D2O):δ 5.11 (d, J=3.2 Hz, 1H), 4.51-4.49 (d, J=8.0 Hz, 1H), 4.42-4.31 (dd, J=13.4, 6.7 Hz, 1H), 4.20-4.12 (m, 2H), 3.79-3.54 (m, 4H), 3.45-3.32 (m, 1H), 2.32-2.27 (s, 3H), 2.24-2.21 (m, 2H), 2.15-1.90 (m, 1H), 1.89 (d, J=25.3 Hz, 1H), 1.59-1.48 (d, 3H), 1.33-1.27 (d, 3H) ppm; ESI-MASS: m/z Calcd. for C₁₉H₃₂N₄O₁₁ 492.3 [M+Na]⁺: 515.3.

Example-3: (4R)-5-amino-4-((2S)-2-((2R)-2-(((3R,4R, 5S, 6R)-3-(cyclopropanecarboxamido)-2, 5-dihydroxy-6-(hydroxymethyl)tetrahydro-2H-pyran-4-yl)oxy)propanamido)propanamido)-5-oxopentanoic acid (15b, 6.2 g, 94%) as a white solid characterized by ¹H NMR, MASS-ESI, ¹H NMR (400 MHz, CD3OD) δ 5.13-5.08 (d, J=3.2 Hz, 1H), 4.52-4.48 (d, J=8.0 Hz, 1H), 4.45-4.37 (dd, J=13.4, 6.7 Hz, 1H), 4.24-4.12 (m, 2H), 3.78-3.57 (m, 4H), 3.42-3.32 (m, 1H), 2.31-2.17 (m, 1H), 2.14-1.99 (m, 1H), 1.90-1.77 (m, 2H), 1.60-1.47 (m, 1H), 1.40-1.25 (m, 6H), 0.84-0.71 (m, 2H), 0.69-0.61 (m, 2H) ppm; ESI-MS: m/z Calcd for C₂₁H₃₄N₄O₁₁ 518.32, found [M+H]⁺ 519, [M+Na]⁺541. [M+K]⁺557.

Example-4: (4R)-5-amino-4-((2S)-2-((2R)-2-(((3R,4R,5S,6R)-3-(cyclobutanecarboxamido)-2,5-dihydroxy-6-(hydroxymethyl)tetrahydro-2H-pyran-4-yl)oxy)propanamido)propanamido)-5-oxopentanoic acid (15c, 6.1 g, 92%) as a white solid characterized by 1H NMR, MASS-ESI, ¹H NMR (400 MHz, CD3OD) δ 5.12-5.07 (d, J=3.2 Hz, 1H), 4.51-4.47 (d, J=8.0 Hz, 1H), 4.44-4.36 (dd, J=13.4, 6.7 Hz, 1H), 4.25-4.11 (m, 2H), 3.77-3.56 (m, 4H), 3.41-3.33 (m, 1H), 3.15-3.13 (m, 1H), 2.31-2.16 (m, 1H), 2.15-1.98 (m, 1H), 1.91-1.76 (m, 2H), 1.62-1.48 (m, 1H), 1.41-1.23 (m, 10H), 0.90-0.75 (m, 2H) ppm; ESI-MS: m/z Calcd for C₂₂H₃₆N₄O₁₁ 532.24, found [M+H]⁺ 534.

Example-5: (4R)-5-amino-4-((2S)-2-((2R)-2-(((3R,4R,5S,6R)-3-(cyclopentanecarboxamido)-2, 5-dihydroxy-6-(hydroxymethyl)tetrahydro-2H-pyran-4-yl)oxy)propanamido)propanamido)-5-oxopentanoic acid (15d, 6.4 g, 95%) as a white solid characterized by ¹H NMR, MASS-ESI, ¹H NMR (400 MHz, CD3OD) δ 5.11-5.06 (d, J=3.2 Hz, 1H), 4.51-4.46 (d, J=8.0 Hz, 1H), 4.43-4.35 (dd, J=13.4, 6.7 Hz, 1H), 4.24-4.11 (m, 2H), 3.76-3.55 (m, 4H), 3.40-3.32 (m, 1H), 2.55-2.35 (m, 2H), 2.30-2.25 (m, 2H), 2.14-2.10 (m, 3H), 2.05-1.47 (m, 6H), 1.44-1.25 (m, 6H), ppm; ESI-MS: m/z Calcd for C₂₃H₃₈N₄O₁₁ 546.25, found [M+K]⁺584.

Example-6: (4R)-5-amino-4-((2S)-2-((2R)-2-(((3R,4R,5S,6R)-3-(cyclohexanecarboxamido)-2,5-dihydroxy-6-(hydroxymethyl)tetrahydro-2H-pyran-4-yl)oxy)propanamido)propanamido)-5-oxopentanoic acid (15e, 6.3 g, 93%) given as example 2 involves all the steps for the synthesis of peptide as given in FIG. 1 of example 1 and the specific sugar fragment prepared as explained in FIG. 2 of example 1 and their successive acid amine coupling and deprotection as given under example 1 and the final compound was obtained as a white solid characterized by ¹H NMR, MASS-ESI, ¹H NMR (400 MHz, CD3OD) δ 5.10-5.05 (d, J=3.2 Hz, 1H), 4.51-4.45 (d, J=8.0 Hz, 1H), 4.42-4.34 (dd, J=13.4, 6.7 Hz, 1H), 4.25-4.10 (m, 2H), 3.75-3.54 (m, 4H), 3.41-3.31 (m, 1H), 2.52-2.45 (m, 2H), 2.34-2.25 (m, 2H), 2.16-1.98 (m, 2H), 1.95-1.74 (m, 3H), 1.71-1.49 (m, 6H), 1.50-1.25 (m, 6H), 1.17-1.10 (m, 2H) ppm; ESI-MS: m/z Calcd for C₂₄H₄₀N₄O₁₁ 560.27, found, [M+K]⁺599.

Example-7:(4R)-5-amino-4-((2S)-2-((2R)-2-(((3R,4R,5S,6R)-2,5-dihydroxy-6-(hydroxymethyl)-3-(8-(tetradecanoyloxy)octanamido)tetrahydro-2H-pyran-4-yl)oxy)propanamido)propanamido)-5-oxopentanoic acid (15f, 7 g, 93%) given as example 2 involves all the steps for the synthesis of peptide as given in FIG. 1 of example 1 and the specific sugar fragment prepared as explained in FIG. 2 of example 1 and their successive acid amine coupling and deprotection as given under example 1 and the final compound was obtained as a white solid characterized by ¹HNMR, MASS-ESI, ¹H NMR (500 MHz, D₂O):δ 7.53 (br, 1H), 5.15-5.08 (m, 1H), 4.55-4.44 (m, 2H), 4.37-4.16 (m, 3H), 4.01-3.45 (m, 5H), 2.57-2.31 (m, 6H), 2.22-2.08 (m, 2H), 1.99-1.78 (m, 4H), 1.75-1.53 (m, 2H), 1.46-1.26 (m, 34H), 1.16 (t, J=7.1 Hz, 3H) ppm; ESI-MS: m/z Calcd. for C₃₉H₇₀N₄O₁₃ 802.5 found [M+H]⁺ 803.6, [M+Na]⁺825.7

Example-8: (4R)-5-amino-4-((2S)-2-((2R)-2-(((3R,4R,5S,6R)-3-(8-(dodecanoyloxy)octanamido)-2,5-dihydroxy-6-(hydroxymethyl)tetrahydro-2H-pyran-4-yl)oxy)propanamido)propanamido)-5-oxopentanoic acid (15 g, 6.8 g, 91%) given as example 2 involves all the steps for the synthesis of peptide as given in FIG. 1 of example 1 and the specific sugar fragment prepared as explained in FIG. 2 of example 1 and their successive acid amine coupling and deprotection as given under example 1 and the final compound was obtained as a white solid characterized by: ¹H NMR (500 MHz, CD₃OD): δ 5.21-5.08 (m, 1H), 4.56-4.42 (m, 3H), 4.38-3.99 (m, 7H), 2.58-2.10 (m, 8H), 1.74-1.25 (m, 34H), 1.10-0.88 (t, J=7.2 Hz, 3H) ppm; ESI-MASS: m/z Calcd. for C₃₇H₆₆N₄O₁₃ 774 [M+H]⁺: 775.

Example-9: (4R)-5-amino-4-((2S)-2-((2R)-2-(((3R,4R,5S,6R)-2,5-dihydroxy-6-(hydroxymethyl)-3-(8-(octanoyloxy)octanamido)tetrahydro-2H-pyran-4-yl)oxy)propanamido)propanamido)-5-oxopentanoic acid (15h, 6.5 g, 90%) given as example 2 involves all the steps for the synthesis of peptide as given in FIG. 1 of example 1 and the specific sugar fragment prepared as explained in FIG. 2 of example 1 and their successive acid amine coupling and deprotection as given under example 1 and the final compound was obtained as a white solid characterized by: ¹H NMR (500 MHz, CD₃OD): δ 5.28-5.10 (m, 1H), 4.56-4.26 (m, 4H), 4.39-3.85 (m, 6H), 2.58-2.10 (m, 8H), 1.75-1.08 (m, 26H), 1.12-0.87 (t, J=7.1 Hz, 3H) ppm; ESI-MASS: m/z Calcd. for C₃₃H₅₈N₄O₁₃ 718 [M+H]⁺: 719.

Example-10:(4R)-5-amino-4-((2S)-2-((2R)-2-(((3R,4R,5S,6R)-2,5-dihydroxy-6-(hydroxymethyl)-3-(tetrahydrofuran-2-carboxamido)tetrahydro-2H-pyran-4-yl)oxy)propanamido)propanamido)-5-oxopentanoic acid(15i, 5.2 g, 91%) given as example 2 involves all the steps for the synthesis of peptide as given in FIG. 1 of example 1 and the specific sugar fragment prepared as explained in FIG. 2 of example 1 and their successive acid amine coupling and deprotection as given under example 1 and the final compound was obtained as a white solid characterized by: ¹H NMR (300 MHz, CD₃OD) δ 5.14-5.06 (d, J=3.2 Hz, 1H), 4.78-4.62 (m, 1H), 4.58-4.45 (d, J=11.9 Hz, 1H), 4.42-4.23 (m, 3H), 4.15-4.06 (m, 1H), 3.86-3.78 (m, 5H), 3.58-3.41 (m, 2H), 2.44-2.27 (m, 2H), 2.23-2.05 (m, 3H), 1.97-1.74 (m, 3H), 1.44-1.23 (dd, J=10.9, 9.7 Hz, 6H); ESI-MASS: m/z Calcd. for C₁₉H₃₂N₄O₁₁ 546.3 [M+Na]⁺: 469.3

### Example-11 Biological Activity: Evaluation of Adjuvant activity

### Sources of Biologicals

Balb-C mice and cell lines for in vivo and in vitro immunopharmacological assay were purchased from the commercial sources. IAEC permission has been obtained for the procurement and use of animals

| Name of the | | | |
|---|---|---|---|
| biological | | | |
| resource | Source of access | Name | Contact Details |
| Balb-C Mice | Centre for | Balb-C Mice | Centre for Cellular & Molecular |
| | Cellular & | IAEC IICT/66/2016 | Biology Habsiguda, Metro |
| | Molecular | | Station, , India. Contact: Animal |
| | Biology, | | House, CSIR-CCMB, Tarnaka, |
| | Hyderabad, | | Hyderabad 500007. CPCSEA Reg. |
| | India | | No. 20/GO/RBi/S/1999/ CPCSEA |
| Recombinant | Purchased from | Polyvalent dengue | Prospec,Protein technologies, |
| antigens | the Prospec, | antigen (DENV), | Israel represented by Sailendra K |
| | Protein | HBsAg surface | Ray, LabproIndia, Plot no26, |
| | technologies, | antigen, and Japanese | Room No. 302, 3^{rd} |
| | Israel | Encephalitis virus | Floor,Nacharam, Hyderabad- |
| | | (JEV) antigen | 500076, Tel 040-27275604 |

Adjuvanticity of 15b was evaluated using antigens (OVA, HBsAg, DENV and JEV) in BALB/c mice model—From the immunized mice retro-orbital sampling or retro-orbital blood was collected on 14th and 28^{th} day before sacrifice. Serum was separated by centrifugation at 12,500 rpm for 5 min. Transferred to a clean centrifuge tube and store at −80 until used.

The plates were coated with antigen in carbonate buffer. Plates were incubated at 4° C. overnight. They were then washed 3 times with PBS/Tween, and non-specific binding sites were blocked by adding 200 μl of blocking solution. Plates were incubated at room temperature for 1 h. Then 3 times wash is done, diluted standards and samples to desired concentrations in blocking solution were added to the plates. Incubate at 37° C. for 1 h or at 4° C. overnight. Plates were washed 3 times with PBS/Tween. Avidin-Horseradish Peroxidase (Av-HRP) was diluted and added. Incubated at room temperature for 30 min. Plates were washed 3 times with PBS/Tween. OPD Substrate was added and plates were incubated at room temperature (4-30 min) for colour development. The colour reaction was stopped by adding 50 μl of stop solution. Optical density (OD) was read at 492 nm.

Serum anti-antigens (OVA, HBsAg, DENV and JEV) IgG titer. antigen specific IgG was assayed by indirect ELISA and titers obtained after booster immunization reveals that the production of anti-antigen antibodies was strongly enhanced in mice treated with conjugates in comparison with antigen alone.

### In Vivo Cytokine Estimation

ELISA was carried out by Bio Legend capture and detection antibodies. Briefly, 96-well plates were coated with capture antibody dissolved in coating buffer per well incubated overnight at 4° C. Wells were blocked with BSA for 1 h at RT. After blocking, 50 μL/well of serum was added and incubated for 3 h. After washing, Biotinylated secondary antibody was added along with enzyme. Plates were incubated for 1 h at RT. Then plates were washed and TMB substrate solution was added. The reaction was stopped after 30 min with a stopping solution. Absorbance was measured at 450 nm with a plate reader.

### Immunophenotyping

Staining for extracellular markers Staining was as per the manufacturer's protocol and run on a BD FACS Verse flow cytometer. Compensation was established using BD Biosciences compensation beads. Post-acquisition flow cytometry analysis was performed using FACS Suite software.

### In Vivo Splenocyte Proliferation

Splenocytes were seeded into 96-well flat-bottom microtiter plates having 1×10⁵ cells/well in 100 mL complete RPMI-1640 medium. Plates were incubated at 37° C. with 5% CO2. After 48 h, 20 mL MTT solution (5 mg/mL) was added to each well and left to incubate for next 4 h. Untransformed MTT (180 mL) was removed from each well by pipetting. A total of 180 mL of DMSO was added and the absorbance was evaluated in an ELISA reader at 630 nm on the multimode reader (Infinite 200 Pro, Switzerland) after 15 min

TABLE 1
In vitro Hemolysis assay

| | % Hemolysis |
|---|---|
| Dis. Water | 100 ± 0.94 |
| PBS | 0 |
| MDP | 0.450128 ± 0.014 |
| 15b | 2.30179 ± 0.147 |

TABLE 2
In vitro splenocyte proliferation assay

| EXVIVO | | CC | | MDP | | 15b | |
|---|---|---|---|---|---|---|---|
| MTT | | ConA | LPS | ConA | LPS | ConA | LPS |
| 100 | μg | 1.00- | 0.9124- | 1.1569- | 1.431967- | 1.10- | 0.9568- |
| | | 2.14 | 1.54 | 1.78 | 1.97 | 1.98 | 1.54 |
| 10 | μg | | | 1.05- | 1.00- | 1.1- | 1.0- |
| | | | | 1.47 | 1.74 | 1.75 | 1.74 |
| 1 | μg | | | 1.02- | 0.95- | 1.08- | 1.02- |
| | | | | 1.76 | 1.84 | 1.74 | 1.46 |

TABLE 3
IgG titer with OVA antigen

| | | | IgG |
|---|---|---|---|
| OVA | 100 | μg | 6400-12800 |
| MDP | 20 | μg | 42666.67-75241.21 |
| 15b | 1 | μg | 1600-124000 |
| | 10 | μg | 204800-416000 |
| | 100 | μg | 115000-320000 |

TABLE 4
Cytokine assay on restimulation of splenocytes with OVA antigen

| | IFN | | |
|---|---|---|---|
| | (pg/ml) | IL-1(pg/ml) | IL-4(pg/ml) |
| 15b | 80.6- | 36.1- | 56.35- |
| 1 μg | 88.24 | 41.95 | 61.47 |
| 15b | 75.06667- | 38.23333- | 25.16667- |
| 10 μg | 82.14 | 45.27 | 31.74 |
| 15b | 68.6- | 23.06667- | 25.1- |
| 100 μg | 75.45 | 29.47 | 29.47 |

TABLE 5
IgG titer with HBsAg antigen HBsAg-15b

| | HBsAg alone | 15b |
|---|---|---|
| 1 μg | | 51200-102400 |
| 10 μg | 25600-34570 | 102400-240000 |

TABLE 6
Post HBsAg immunization In vivo splenocyte proliferation assay

| | Cells alone | HBsAg | LPS | Con A |
|---|---|---|---|---|
| HBsAg alone | 100 | 100 | 100 | 100 |
| 20 | 130.6- | 131.87- | 111.6- | 163.56- |
| μg | 145.74 | 140.24 | 124.58 | 175.38 |
| 15b | 123.5- | 125.07- | 165.8- | 176.5- |
| 1 μg | 142.87 | 135.87 | 175.34 | 181.54 |
| 15b | 115.5- | 124.30- | 151.66- | 188.42- |
| 10 μg | 125.76 | 134.84 | 162.47 | 194.28 |
| 15b | 142.1- | 141.19- | 164.9- | 173.3- |
| 100 μg | 149.25 | 148.27 | 175.24 | 149.24 |

TABLE 7
Post HBsAg immunization In vivo cytokine assay

| | IFN | IL-1 | IL-4 | IL-6 | IL-10 |
|---|---|---|---|---|---|
| | (pg/ml) | (pg/ml) | (pg/ml) | (pg/ml) | (pg/ml) |
| HBSAG | 75.28- | 12.43- | 22.43- | 93.23- | 25.5- |
| | 84.28 | 19.47 | 34.78 | 110.87 | 31.84 |
| MDP | 97.28- | 33.6- | 22.63- | 207.3- | 49- |
| | 110.54 | 37.48 | 27.58 | 278-28 | 55.84 |
| 15b | 80.6 | 16.6- | 23.73- | 137.83- | 22.7- |
| 1 μg | | 20.75 | 29.48 | 145.89 | 29.48 |
| 15b | 75.06667 | 19.23- | 30.46- | 197.2- | 22.33- |
| 10 μg | | 28.47 | 39.27 | 201.48 | 29.48 |
| 15b | 68.6 | 29.86- | 25.8- | 380.85- | 30.63- |
| 100 μg | | 37.48 | 34.87 | 395.75 | 38.54 |

TABLE 8
Post HBsAg immunization Immunophenotyping

| | CD4 | CD8 |
|---|---|---|
| HBsAg | 21.64-28.47 | 10.26-14.38 |
| MDP | 17.11-19.48 | 5.9-9.57 |
| 15b -1 μG | 24.48-29.48 | 12.71-19.48 |
| 15b -10 μG | 25.47-29.48 | 12.67-18.54 |

TABLE 9
IgG titer with Dengue

| | | MDP | |
|---|---|---|---|
| IgG | DENGUE | (20 μg) | 15b |
| 1 μg | 40000- | 160000- | 80000- |
| | 80000 | 240000 | 160000 |
| 10 μg | | | 640000- |
| | | | 950000 |

TABLE 10
Post Denv immunization in vivo cytokine assay

| | IL-1 | IL-2 | IL-6 | IL-10 | TNF |
|---|---|---|---|---|---|
| | (pg/ml) | (pg/ml) | (pg/ml) | (pg/ml) | (pg/ml) |
| Dengue | 2.65- | 15.1- | 346.44- | 44.95- | 27.56- |
| alone | 5.95 | 19.47 | 374.47 | 49.28 | 34.28 |
| MDP | 7.25- | 35.65- | 203.36- | 75.8- | 55.5- |
| 20 μg | 9.48 | 39.27 | 227.15 | 89.27 | 60.27 |
| 15b | 18.05- | 5- | 297.08- | 55.2- | 15.5- |
| 1 μg | 20.48 | 9 | 304.84 | 61.28 | 21.48 |
| 15b | 8.75- | 9.25- | 257.50- | 49.7- | 32.8- |
| 10 μg | 10.57 | 10.84 | 274.58 | 54.84 | 47.28 |

TABLE 11
Post Denv immunization In vivo splenocyte proliferation assay

| | Cells alone | Cells | Cells + LPS | Cells + Con-A |
|---|---|---|---|---|
| UT | 100 | 100 | 100 | 100 |
| Dengue antigen | 111.69- | 120.15- | 115.63- | 96.25- |
| | 115.24 | 125.48 | 119.24 | 104.57 |
| MDP | 104.48- | 110.45- | 115.6- | 123.53- |
| | 109.54 | 115.48 | 119.84 | 128.48 |
| 15b | 99.26- | 111.72- | 122.4- | 115.61- |
| 1 μg | 104.65 | 116.24 | 127.35 | 120.48 |
| 15b | 115- | 121.72- | 126.9- | 136.72- |
| 10 μg | 119 | 127.24 | 134.85 | 140.27 |

TABLE 12
Post Denv immunization Immunophenotyping

| | CD4 | CD8 | CD19 | CD80 | CD 28 |
|---|---|---|---|---|---|
| DEV | 34.37-39.5 | 22.03-27.48 | 33.92-39.48 | 5.2-9.8 | 3.3-8.4 |
| MDP | 41.25-46.5 | 16.39-21.84 | 39.65-42.57 | 4.42-7.5 | 5.33-9.4 |
| 15b -1 μG | 36.26-41.8 | 22.26-29.48 | 30.57-35.48 | 6.3-8.4 | 3.65-7.48 |
| 15b -10 μG | 38.74-43.84 | 22.92-27.84 | 36.19-41.28 | 6.11-11.4 | 3.68-8.41 |

TABLE 13
IgG titer with JEV antigen

| | JEV alone | MDP-20 μg | 15b |
|---|---|---|---|
| 1 μg | | | 64000-128000 |
| 10 μg | 16000-32000 | 128000-320000 | 8000-16000 |
| 100 μg | | | 40000-80000 |

TABLE 14
Post JEV immunization In vivo cytokine assay

| | IL-1 | IL-2 | IFN | IL-6 | IL-10 |
|---|---|---|---|---|---|
| | (pg/ml) | (pg/ml) | (pg/ml) | (pg/ml) | (pg/ml) |
| JEV alone | 62.7-8.41 | 15.0-20.41 | 1.9-2.4 | 256.8-274.01 | 63.7-64.24 |
| MDP 20 μg | 62.8-67.54 | 13.88-19.47 | 5.01-7.37 | 149.2-154.27 | 44.96-47.58 |
| 15b 1 μg | 63.369.48 | 13.77-19.81 | 4.21-6.37 | 157.05-160.47 | 48-51.02 |
| 15b 10 μg | 61.9-67.25 | 12.17-15.34 | 11.03-13.05 | 233.3-245.27 | 52.3-54.28 |
| 15b 100 μg | 70.1-73.48 | 23.97-27.45 | 3.06-6.48 | 279.85-281.47 | 57.5-59.48 |

TABLE 15
Post JEV immunization In vivo splenocyte proliferation assay

| | Normal | Restimulated | LPS | Con A |
|---|---|---|---|---|
| UT | 100 | 100 | 100 | 100 |
| JEV alone | 109.84-111.24 | 117.9-120.48 | 106.3-109.54 | 134.4-139.27 |
| MDP 20 μg | 98.56-101.02 | 93.3-97.28 | 85.1-89.24 | 141.95-144.28 |
| 15b 1 μg | 118.6-120.48 | 116.5-119.27 | 118.2-120.01 | 117.5-119.48 |
| 15b 10 μg | 111.38-115.78 | 114.2-117.24 | 104.6-109.57 | 124.55-126.85 |

TABLE 16
Post JEV immunization Immunophenotyping

| | CD4 | CD8 |
|---|---|---|
| JEV | 40.78-45.27 | 16.31-19.24 |
| MDP | 43.45-48.28 | 19.05-21.48 |
| 15b -1 μG | 39.75-42.58 | 14.56-19.54 |
| 15b -10 μG | 41.86-43.28 | 14.78-17.25 |

Table-17 and Table 18: Immunopharmacological evaluation of compound 15c and 15 d carried out using OVA antigen as per protocols described in Example-10.

TABLE 17

| | | IgG |
|---|---|---|
| OVA | 100 μg | 6400-12800 |
| MDP | 20 μg | 42666.67-75241.21 |
| 15c | 1 μg | 25000-102400 |
| | 10 μg | 10000-85000 |
| | 100 μg | 5500-75000 |

TABLE 18

| | | IgG |
|---|---|---|
| OVA | 100 μg | 6400-12800 |
| MDP | 20 μg | 42666.67-75241.21 |
| 15d | 1 μg | 3200-12400 |
| | 10 μg | 3200-41600 |
| | 100 μg | 6400-84000 |

Table-19 and Table-20: Immunopharmacological evaluation
of compound 15e and 15f was carried out using OVA
antigen using protocols as described in Example-10.

| | | IgG |
|---|---|---|
| OVA | 100 μg | 6400-12800 |
| MDP | 20 μg | 42666.67-75241.21 |
| 15e | 1 μg | 1600-12400 |
| | 10 μg | 1600-41600 |
| | 100 μg | 12800-96000 |
| | | IgG |
| OVA | 100 μg | 6400-12800 |
| MDP | 20 μg | 42666.67-75241.21 |
| 15f | 1 μg | 8000-25400 |
| | 10 μg | 5000-41600 |
| | 100 μg | 200000-960000 |

TABLE 21
Immunopharmacological evaluation of compound 15f was carried out
with JEV antigen using protocols as described in Example-10.

| IgG | Titer |
|---|---|
| JEV | 40000-49000 |
| MDP | 160000-320000 |
| 15f-5 μG | 80000-160000 |
| 15f -20 μG | 160000-640000 |
| 15f -50 μG | 640000-9560000 |

TABLE 22
Immunophenotyping post JEV immunization

| | IL-1 | IL-2 | IL-4 | IL-10 | IL-12 |
|---|---|---|---|---|---|
| | (pg/ml) | (pg/ml) | (pg/ml) | (pg/ml) | (pg/ml) |
| JEV | 545-625 | 45-60 | 90-110 | 349-374 | 95-110 |
| MDP | 1467-1725 | 89-97 | 145-157 | 2000-2500 | 140-154 |
| 15f-5 μG | 745-845 | 41-52 | 134-155 | 243-294 | 124-147 |
| 15f-20 μG | 1059-1124 | 52-64 | 138-146 | 398-421 | 175-194 |
| 15f-50 μG | 1487-1634 | 69-76 | 148-164 | 424-487 | 136-147 |

TABLE 23
In vitro splenocyte proliferation assay

| | % |
|---|---|
| MDPO Myr | Proliferation |
| 1000 μG/ml | 65.24 |
| 500 μG/ml | 95.87 |
| 250 μG/ml | 120.54 |
| 125 μG/ml | 135.24 |

TABLE 24
Post immunization In vivo cytokine assay

| | IL-1 | IL-2 | IL-4 | IL-10 | IL-12 | TNF |
|---|---|---|---|---|---|---|
| | (pg/ml) | (pg/ml) | (pg/ml) | (pg/ml) | (pg/ml) | (pg/ml) |
| JEV | 545-625 | 45-60 | 90-110 | 349-374 | 95-110 | 545-672 |
| MDP | 1467-1725 | 89-97 | 145-157 | 2000-2500 | 140-154 | 645-694 |
| 15f-5 | 745-845 | 41-52 | 134-155 | 243-294 | 124-147 | 624-654 |
| μG | | | | | | |
| 15f-20 | 1059-1124 | 52-64 | 138-146 | 398-421 | 175-194 | 898-942 |
| μG | | | | | | |
| 15f-50 | 1487-1634 | 69-76 | 148-164 | 424-487 | 136-147 | 1024-1124 |
| μG | | | | | | |

TABLE 25
In vivo splenocyte proliferation assay

| | JEV | LPS | Con A |
|---|---|---|---|
| JEV | 120-135 | 140-155 | 110-124 |
| MDP | 145-155 | 189-194 | 194-210 |
| 15f-5 μG | 139-147 | 124-134 | 125-141 |
| 15f-20 μG | 149-162 | 128-137 | 134-142 |
| 15f-50 μG | 155174 | 184-192 | 245-264 |

Table-26 and Table-27: Immunopharmacological evaluation of compound 15 g and 15h was carried out with OVA antigen using biological resources and protocols as described in Example-10.

TABLE 26

| | | IgG |
|---|---|---|
| OVA | 100 μg | 6400-12800 |
| MDP | 20 μg | 42666.67-75241.21 |
| 15g | 1 μg | 25000-124000 |
| | 10 μg | 10000-60000 |
| | 100 μg | 5000-35000 |

TABLE 27

| | | | IgG |
|---|---|---|---|
| OVA | 100 | μg | 6400-12800 |
| MDP | 20 | μg | 42666.67-75241.21 |
| 15 h | 1 | μg | 8750-64000 |
| | 10 | μg | 10000-50000 |
| | 100 | μg | 3750-25000 |

TABLE-28
Immunopharmacological evaluation of compound 15i was
carried out with OVA antigen using biological resources
and protocols as described in Example-10.

| | | IgG |
|---|---|---|
| OVA | 100 μg | 6400-12800 |
| MDP | 20 μg | 42666.67-75241.21 |
| 15i | 1 μg | 4800-50000 |
| | 10 μg | 102400-560000 |
| | 100 μg | 119466-484000 |

From all the above in vitro and in vivo immunological evaluation data of 15b against Ovalbumin, DEV, JEV and HBsAg antigens, and 15f against JEV antigen, it could be concluded that the adjuvant molecule was non-toxic and non-hemolytic even at higher concentrations and has elicited a significant antibody titer, along with cytokine production against most of the antigens tested. A potent humoral and cell mediated immune response was observed which is very much required characteristic for an effective adjuvant.

In an embodiment the present invention also provides a prophylactic vaccine composition comprising: a muramyl dipeptide compound of general formula-I and an antigen is selected from inactivated or live attenuated infectious pathogens their subunit, either natural pathogen derived or recombinant, a conjugate vaccine antigen or a combination thereof.

The following vaccine compositions with muramyl dipeptide compound of general formula-I are prepared wherein the ratio of antigen: muramyl dipeptide compound is 1:0.1-1.

Vaccine compositions

DEV and muramyl dipeptide compound
JEV and muramyl dipeptide compound
HBsAg and muramyl dipeptide compound

1-10. (canceled)

11. A muramyl dipeptide compound according to general formula (I): 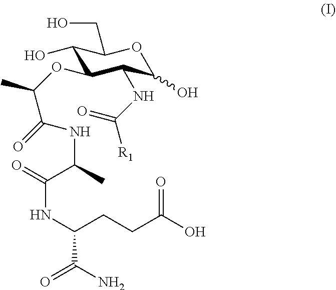
where:
R₁ is selected from substituted or unsubstituted (C₃-C₆)cycloalkyl, 3 to 6 membered heterocycloalkyl with one or more hetero atoms selected from nitrogen and oxygen, or 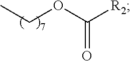
and
R₂ is a C₁ to C₁₈ aliphatic chain.

12. The muramyl dipeptide compound according to claim 11, wherein R₁ is selected from the group consisting of

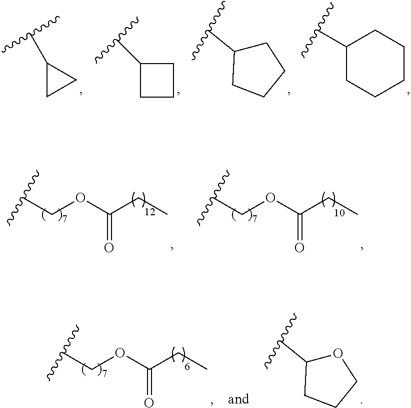

13. The muramyl dipeptide compound according to claim 11, selected from group consisting of
(4R)-5-amino-4-((2S)-2-((2R)-2-(((3R,4R,5S,6R)-3-(cyclopropanecarboxamido)-2,5-dihydroxy-6-(hydroxymethyl)tetrahydro-2H-pyran-4-yl)oxy)propanamido)propanamido)-5-oxopentanoic acid;
(4R)-5-amino-4-((2S)-2-((2R)-2-(((3R,4R,5S,6R)-3-(cyclobutanecarboxamido)-2,5-dihydroxy-6-(hydroxymethyl)tetrahydro-2H-pyran-4-yl)oxy)propanamido)propanamido)-5-oxopentanoic acid;
(4R)-5-amino-4-((2S)-2-((2R)-2-(((3R,4R,5S,6R)-3-(cyclopentanecarboxamido)-2,5-dihydroxy-6-(hydroxymethyl)tetrahydro-2H-pyran-4-yl)oxy)propanamido)propanamido)-5-oxopentanoic acid;
(4R)-5-amino-4-((2S)-2-((2R)-2-(((3R,4R,5S,6R)-3-(cyclohexanecarboxamido)-2,5-dihydroxy 6-(hydroxymethyl)tetrahydro-2H-pyran-4-yl)oxy)propanamido)propanamido)-5-oxopentanoic acid;
(4R)-5-amino-4-((2S)-2-((2R)-2-(((3R,4R,5S,6R)-2,5-dihydroxy-6-(hydroxymethyl)-3-(8-(tetradecanoyloxy)octanamido)tetrahydro-2H-pyran-4-yl)oxy)propanamido)propanamido)-5-oxopentanoic acid;
(4R)-5-amino-4-((2S)-2-((2R)-2-(((3R,4R,5S,6R)-3-(8-(dodecanoyloxy)octanamido)-2,5-dihydroxy-6-(hydroxymethyl)tetrahydro-2H-pyran-4-yl)oxy)propanamido)propanamido)-5-oxopentanoic acid;
(4R)-5-amino-4-((2S)-2-((2R)-2-(((3R,4R,5S,6R)-2,5-dihydroxy-6-(hydroxymethyl)-3-(8-(octanoyloxy)octanamido)tetrahydro-2H-pyran-4-yl)oxy)propanamido)propanamido)-5-oxopentanoic acid; and
(4R)-5-amino-4-((2S)-2-((2R)-2-(((3R,4R,5S,6R)-2,5-dihydroxy-6-(hydroxymethyl)-3-(tetrahydrofuran-2-carboxamido)tetrahydro-2H-pyran-4-yl)oxy)propanamido)propanamido)-5-oxopentanoic acid.

14. An advanced intermediate of the muramyl dipeptide compound according to claim 11, comprising O-benzyl protecting groups on D-glutamic acid, an O-benzyl on an anomeric hydroxyl group, and a benzylidine protecting group on a 5,6 diol of a sugar moiety, the advanced intermediate having formula (II): 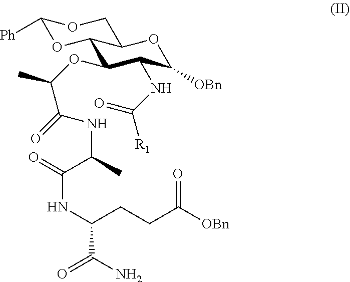
where:
R₁ is selected from substituted or unsubstituted (C₃-C₆)cycloalkyl, 3 to 6 membered heterocycloalkyl with one or more hetero atoms selected from nitrogen and oxygen, or 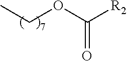
where R₂ is a C₁ to C₁₈ aliphatic chain.

15. A process for preparing the muramyl dipeptide compound according to claim 11, the process comprising:
(a) performing a diazotransfer of a compound (7): 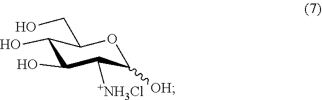
with trifluoromethanesulfonic azide to obtain (3R,4R,5S,6R)-3-azido-6-(hydroxymethyl)tetrahydro-2H-pyran-2,4,5-triol as a compound (8): 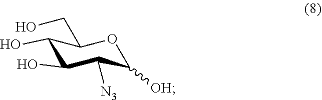
(b) performing an anomeric benzylation of the compound (8) in the presence of a Lewis acid to obtain (2R,3S,4R,5R,6S)-5-azido-6-(benzyloxy)-2-(hydroxymethyl)tetrahydro-2H-pyran-3,4-diol as a compound (9): 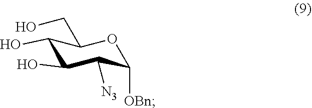
(c) performing a benzylidene protection of the compound (9) in the presence of an organic acid with benzaldehyde dimethylacetal to obtain (2R,4aR,6S,7R,8R,8aS)-7-azido-6-(benzyloxy)-2-phenylhexahydropyrano[3,2-d][1,3]dioxin-8-ol as a compound (10): 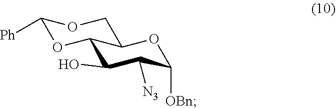
(d) performing an O-alkylation of the compound (10) in the presence of a metal hydride with S-(2)-chloropropionic acid to obtain (R)-2-(((2R,4aR,6S,7R,8R,8aS)-7-azido-6-(benzyloxy)-2-phenylhexahydropyrano[3,2-d][1,3]dioxin-8-yl)oxy)propanoic acid as a compound (11): 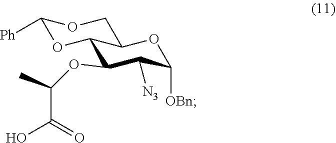
(e) peptide coupling the compound (11) with L-alanyl-D-isoglutamine benzyl ester trifluoroacetate in the presence of a first coupling agent and a first base to obtain (R)-benzyl 5-amino-4-((S)-2-((R)-2-(((2R,4aR,6S,7R,8R,8aS)-7-azido-6-(benzyloxy)-2-phenylhexahydropyrano[3,2-d][1,3]dioxin-8-yl)oxy)propanamido)propanamido)-5-oxopentanoate as a compound (12): 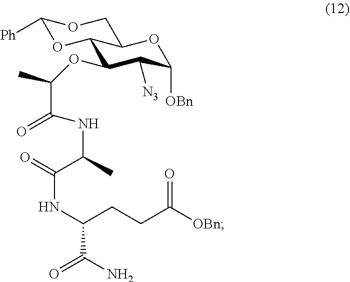
(f) reducing the compound (12) with triphenylphosphine to obtain benzyl (R)-5-amino-4-((S)-2-((R)-2-(((2R,4aR,6S,7R,8R,8aS)-7-amino-6-(benzyloxy)-2-phenylhexahydropyrano[3,2-d][1,3]dioxin-8-yl)oxy)propanamido)propanamido)-5-oxopentanoate as a compound (13): 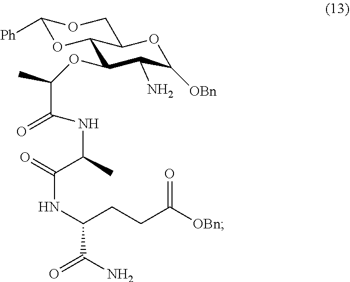
(g) acid and amine coupling the compound (13) with a cycloalkyl carboxylic acid or mixed ester carboxylic acid C(O)R₁ where R₁ is selected from substituted or unsubstituted (C₃-C₆) cycloalkyl, 3 to 6 membered heterocvcloalkyi with one or more hetero atoms selected from nitrogen and oxygen, or 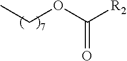
where R₂ is a C₁ to C₁₈ aliphatic ester, in the presence of a second coupling agent and a second base to obtain a compound (14): 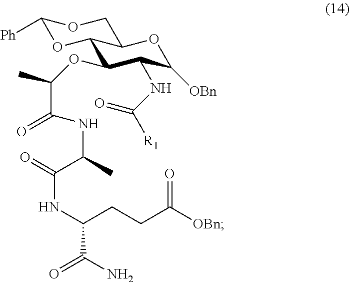
and
(h) performing a hydrogenolysis of the compound (14) to obtain the muramyl dipeptide compound.

16. The process according to claim 15, wherein the Lewis acid of (b) is selected from the group consisting of BF₃, AlCl₃, ZnCl₂, p-toluenesulfonic acid, chlorosulfonic acid, camphorsulfonic acid, HCl, acetyl chloride, Amberlite and zeolite, and clay.

17. The process according to claim 15, wherein the acid of (c) is selected from the group consisting of p-toluenesulfonic acid, camphorsulfonic acid, and ZnCl₂.

18. The process according to claim 15, wherein the metal hydride of (d) is selected from the group consisting of sodium hydride, potassium hydride, and calcium hydride.

19. The process according to claim 15, wherein:
the first coupling agent of (e) and the second coupling agent of (g) are selected from the group consisting of EDCI/HOBt, DCC/DMAP, DIC/DMAP, and T3P; and
the first base in (e) and the second base in (g) are diisopropylethylamine.

20. A prophylactic vaccine composition comprising:
a muramyl dipeptide compound according to claim 11; and
an antigen selected from inactivated or live attenuated infectious pathogens or subunits thereof either recombinant or derived from a natural pathogen, a conjugate vaccine antigen, or a combination thereof.
